(19) United States
(12) Patent Application Publication
(10) Pub. No.: US 2019/0189293 A1
(43) Pub. Date: Jun. 20, 2019

(54) SYSTEM AND METHOD FOR REMOTE PROVISION OF HEALTHCARE

(71) Applicants: Edmund Yee-Lai TSE, Brisbane (AU); Guruparan IYNGKARAN, Brisbane (AU)

(72) Inventors: Edmund Yee-Lai TSE, Brisbane (AU); Guruparan IYNGKARAN, Brisbane (AU)

(21) Appl. No.: 16/326,690

(22) PCT Filed: Aug. 9, 2017
(86) PCT No.: PCT/AU2017/050837

§ 371 Date: Feb. 19, 2019

(30) Foreign Application Priority Data
Aug. 16, 2016 (AU) 2016903248

(51) Int. Cl. G16H 80/00; G16H 15/00; G16H 20/10; G16H 10/60; G16H 50/20

(52) U.S. Cl. CPC G16H 80/00; G16H 15/00; G16H 50/20; G16H 10/60; G16H 20/10

(57) ABSTRACT

A healthcare provision and management system comprises a first electronic device, the first electronic device configured and operable to receive information about a patient; a server, the server configured and operable to receive the information from the first electronic device over a network; a control module, the control module being included in, and/or forming a part of, the server, the control module being configured to control the server to analyse the received information and, based on the received information, generate a draft treatment plan for the patient; and a second electronic device, the second electronic device configured and operable to receive the draft treatment plan and review, revise, and/or authorise the draft treatment plan to produce a finalized treatment plan. The control module is further configured to control the server to analyse the finalized treatment plan and generate therefrom one or more treatment plan implementation calendars each containing one or more events.

### FIELD OF INVENTION

The present invention relates to systems, methods, and processes for providing healthcare remotely. The present invention has particular but not exclusive application in the remote treatment, care, and management of patients with a chronic disease.

### BACKGROUND OF THE INVENTION

The care of patients with chronic diseases is typically shared between a number of stakeholders, who together make up a care unit. A care unit is generally comprised of the Patient, the Patient's General Practitioner Doctor (hereinafter referred to as the “GP”), and the Patient's Specialist Doctor (hereinafter referred to as the “Specialist”). Additional members of the care unit may also include a nurse supporting the GP (hereinafter referred to as the “GP Nurse”), a nurse supporting the Specialist (hereinafter referred to as the “Specialist Nurse”), and/or one or more other healthcare professionals (e.g. pathologist, radiologist, other Specialist doctor, laboratory technician, radiographer, physiotherapist, etc.).

Care, treatment, and management of a patient with a chronic disease are conventionally effected in the following manner:
1. The chronic disease is first diagnosed by the patient's GP.
2. The GP refers the patient to a Specialist for treatment.
3. The Specialist creates an initial treatment plan to treat the disease.
4. The Specialist works with the patient to implement the initial treatment plan.
5. Upon the patient attaining a stable condition, the Specialist creates a second treatment plan to further treat and manage the disease.
6. The Specialist works with the patient to implement the second treatment plan.

In the above conventional manner, steps (4) and (6) are typically performed by the Specialist. The performance of steps (4) and/or (6) by the Specialist is however an ineffective use of the Specialist's time, knowledge, and experience, and should ideally be performed by the GP, GP Nurse, Specialist Nurse, and/or other healthcare professional.

However, due to a variety of factors including a lack of confidence by the GP, GP Nurse, and/or healthcare professional in themselves, a lack of confidence in the GP by the Specialist, and/or a lack of confidence in the GP by the patient, in being able to implement the treatment plan, the ideal arrangement where the patient's GP, GP Nurse, and/or other healthcare professional works with the patient to implement the first and second treatment plans is rarely achievable or achieved.

Additionally, steps (2) to (6) typically involve a significant amount of in-person meetings and appointments, many of which are with the Specialist and yet predominantly serve a purpose of merely checking on the Patient's treatment progress. Such in-person meetings and appointments are again cost ineffective and time consuming, for both the Specialist and the Patient.

There is therefore a need for a system, method, and process to, as much as possible, effect, facilitate and make achievable the generation, management, and execution of a treatment plan by members of a Patient's care unit other than the Specialist. There is further a need for a system, method, and process to, as much as possible, allow this generation, management, and execution to be performed remotely.

### OBJECT OF THE INVENTION

It is one object of the present invention to provide a system, method, and process for shifting responsibilities and tasks related to the generation, management, and execution of a Patient's treatment plan to a GP, GP Nurse, Specialist Nurse, Patient, and/or other healthcare professional without compromising the standard of care nor level of compliance by the Patient to the treatment plan.

It is a further object of the present invention to provide a system, method, and/or process for generating, managing, and executing a treatment plan remotely.

It is still a further object of the present invention is to enable the GP, GP Nurse, Specialist Nurse and/or other healthcare professionals to create medically appropriate draft treatment plans for managing and treating a specialist-treated disease.

### SUMMARY OF THE INVENTION

According to one aspect of the present invention, a healthcare provision and management system comprises a first electronic device, the first electronic device configured and operable to receive information about a patient; a server, the server configured and operable to receive the information from the first electronic device over a network; a control module, the control module being included in, and/or forming a part of, the server, the control module being configured to control the server to analyse the received information and, based on the received information, generate a draft treatment plan for the patient; and a second electronic device, the second electronic device configured and operable to receive the draft treatment plan and review, revise, and/or authorise the draft treatment plan to produce a finalized treatment plan. The control module is further configured to control the server to analyse the finalized treatment plan and generate therefrom one or more treatment plan implementation calendars each containing one or more events.

In one form, the system further comprises a third electronic device, the third electronic device configured and operable to periodically receive from the patient further information about the patient.

In one form, the system further comprises a fourth electronic device, the fourth electronic device configured and operable to review and revise the one or more treatment plan implementation calendars.

Preferably, the information received by the first electronic device includes answers provided in response to a specific question set, and the first electronic device is configured to generate an answer set from the answers; the answer set represents a combination of answers which identifies and is logically associated with a treatment plan template; and the control module generates the draft treatment plan using the associated treatment plan template.

In one form, the further information is received by the third electronic device from health sensors monitoring the patient.

In one form, the further information is received by the third electronic device by way of journal entries entered by the patient through the third electronic device.

In one form, the first electronic device is operated by a general practitioner doctor, the second electronic device is operated by a specialist doctor, the third electronic device is operated by the patient, and the fourth electronic device is operated by a specialist nurse, and the control module is configured to generate a treatment plan implementation calendar that is specific for each of the first, second, third, and fourth electronic devices.

Preferably, the fourth electronic device is configured and operable to review and revise all treatment plan implementation calendars.

Preferably, one or more of the first, second, and fourth electronic devices is operable to generate an analysis parameter for analysing the further information about the patient, and the control module is operable to monitor the further information about the patient and determine if any of the further information satisfies the analysis parameter.

Preferably, the control module performs a triggered revision of one or more of the treatment plan implementation calendars upon determining that a piece of the further information satisfies the analysis parameter.

Preferably, the control module sends a message or notification to one or more of the first, second, and fourth electronic devices upon determining that a piece of the further information satisfies the analysis parameter.

Preferably, one or more of the first, second, and fourth electronic devices is operable to generate a search parameter for searching for test results or test requests, and the control module is operable to search one or more databases for test results or test requests satisfying the search parameter.

In one form, the control module revises one or more treatment plan implementation calendars upon identifying a test result or test request satisfying the search parameter.

In one form, the treatment plan implementation calendar specific for the first electronic device is generated to include one or more of:
(a) Dates and timings for checking in with the patient;
(b) Dates and timing for receiving reports from one or more of the specialist doctor, patient, or specialist nurse;
(c) Dates and timings for sending reports to one or more of the specialist doctor, patient, or specialist nurse;
(d) Dates and timings of patient appointments;
(e) Dates and timings of test results due from the patient;
(f) Dates and timings for the issuance of medication prescription scripts;
(g) Dates and timings for the issuance of test requests;
(h) Partially pre-filled draft prescription scripts; and
(i) Partially pre-filed test requests.

In one form, the treatment plan implementation calendar specific for the second electronic device is generated to include one or more of:
(a) Dates and timings for checking in with the patient;
(b) Dates and timing for receiving reports from one or more of the specialist doctor, patient, or specialist nurse;
(c) Dates and timings for sending reports to one or more of the specialist doctor, patient, or specialist nurse;
(d) Dates and timings of patient appointments;
(e) Dates and timings of test results due from the patient;
(f) Dates and timings for the issuance of medication prescription scripts;
(g) Dates and timings for the issuance of test requests;
(h) Partially pre-filled draft prescription scripts; and
(i) Partially pre-filed test requests.

In one form, the treatment plan implementation calendar specific for the third electronic device is generated to include one or more of:
(a) Dates and timings for drug doses;
(b) Information regarding drugs;
(c) Dates and timings for tests;
(d) Dates and timings for filling medication scripts;
(e) Dates and timings of appointments with the specialist doctor, specialist nurse, and/or general practitioner doctor;
(f) Dates, timings and contents of entries to be made into one or more patient journals;
(g) Prescription scripts; and
(h) Test requests.

In one form, the treatment plan implementation calendar specific for the fourth electronic device is generated to include one or more of:
(a) Dates and timings for checking in with the patient;
(b) Dates and timing for receiving reports from one or more of the specialist doctor, patient, or general practitioner doctor;
(c) Dates and timings for sending reports to one or more of the specialist doctor, patient, or general practitioner doctor;
(d) Dates and timings of patient appointments;
(e) Dates and timings of test results due from the patient;
(f) Dates and timings for the issuance of medication prescription scripts;
(g) Dates and timings for the issuance of test requests;
(h) Partially pre-filled draft prescription scripts; and
(i) Partially pre-filed test requests.

Preferably, the first electronic device is further operable to generate a treatment plan template, a question set, and one or more answer sets which each represent one combination of answers to the question set, and the server is operable to store the treatment plan template in association with at least one of the answer sets, such that the associated answer set(s) identify the treatment plan template.

Preferably, the finalized treatment plan includes an event for setting up a patient journal, and the control module is operable to set up a patient journal accessible by the third electronic device and adapted for input into thereby.

In one form, the event for setting up the patient journal is pre-specified by a treatment plan template on which the finalized treatment plan is based.

In one form, the event for setting up the patient journal is added to the finalized treatment plan by the control module in response to an analysis conducted by the control module on the further information about the patient.

Preferably, one or more of the first electronic device, second electronic device, third electronic device, and fourth electronic device is operable to set up a patient journal accessible by the third electronic device and adapted for input into thereby.

In one form, input into the patient journal is analysed by the server under control of the control module, and in accordance with one or more analysis parameters.

According to another aspect of the present invention, a healthcare provision and management method comprises receiving information about a patient; analysing the received information and, from the analysis of the received information, automatically identifying a treatment plan template from which to generate a draft treatment plan for the patient; generating a draft treatment plan from the identified treatment plan template; reviewing, revising, and/or authorising the draft treatment plan to produce a finalized treatment plan; and analysing the finalized treatment plan to automatically generate therefrom one or more treatment plan implementation calendars each containing one or more events.

Preferably, the method further comprises periodically receiving further information about the patient, and reviewing and/or revising the one or more treatment plan implementation calendars in response to the received further information.

In one form, the information about the patient includes an answer set provided in response to a specific question set, and the answer set represents a combination of answers that is used to automatically identify the treatment plan template.

In one form, the further information is received from health sensors monitoring the patient.

In one form, the further information is received by way of journal entries entered by the patient.

In one form, the one or more automatically generated treatment plan implementation calendars are each generated to be specific for a GP, Specialist, Specialist Nurse, and Patient making up a care unit of the Patient.

Preferably, the method further comprises generating an analysis parameter for analysing the further information about the patient, and monitoring the further information about the patient to determine if any of the further information satisfies the analysis parameter.

Preferably, the method further comprises performing a revision of one or more of the treatment plan implementation calendars upon determining that a piece of the further information satisfies the analysis parameter.

Preferably, the method further comprises sending a message or notification upon determining that a piece of the further information satisfies the analysis parameter.

Preferably, the method further comprises generating a search parameter for searching for test results or test requests, and searching one or more databases for test results or test requests satisfying the search parameter.

Preferably, the method further comprises revising one or more treatment plan implementation calendars upon identifying a test result or test request satisfying the search parameter.

In one form, the treatment plan implementation calendar specific for GP is generated to include one or more of:
(a) Dates and timings for checking in with the patient;
(b) Dates and timing for receiving reports from one or more of the specialist doctor, patient, or specialist nurse;
(c) Dates and timings for sending reports to one or more of the specialist doctor, patient, or specialist nurse;
(d) Dates and timings of patient appointments;
(e) Dates and timings of test results due from the patient;
(f) Dates and timings for the issuance of medication prescription scripts;
(g) Dates and timings for the issuance of test requests;
(h) Partially pre-filled draft prescription scripts; and
(i) Partially pre-filed test requests.

In one form, the treatment plan implementation calendar specific for the Specialist is generated to include one or more of:
(a) Dates and timings for checking in with the patient;
(b) Dates and timing for receiving reports from one or more of the specialist doctor, patient, or specialist nurse;
(c) Dates and timings for sending reports to one or more of the specialist doctor, patient, or specialist nurse;
(d) Dates and timings of patient appointments;
(e) Dates and timings of test results due from the patient;
(f) Dates and timings for the issuance of medication prescription scripts;
(g) Dates and timings for the issuance of test requests;
(h) Partially pre-filled draft prescription scripts; and
(i) Partially pre-filed test requests.

In one form, the treatment plan implementation calendar specific for the Patient is generated to include one or more of:
(a) Dates and timings for drug doses;
(b) Information regarding drugs;
(c) Dates and timings for tests;
(d) Dates and timings for filling medication scripts;
(e) Dates and timings of appointments with the specialist doctor, specialist nurse, and/or general practitioner doctor;
(f) Dates, timings and contents of entries to be made into one or more patient journals;
(g) Prescription scripts; and
(h) Test requests.

In one form, the treatment plan implementation calendar specific for the Specialist Nurse is generated to include one or more of:
(a) Dates and timings for checking in with the patient;
(b) Dates and timing for receiving reports from one or more of the specialist doctor, patient, or general practitioner doctor;
(c) Dates and timings for sending reports to one or more of the specialist doctor, patient, or general practitioner doctor;
(d) Dates and timings of patient appointments;
(e) Dates and timings of test results due from the patient;
(f) Dates and timings for the issuance of medication prescription scripts;
(g) Dates and timings for the issuance of test requests;
(h) Partially pre-filled draft prescription scripts; and
(i) Partially pre-filed test requests.

In one form, the finalized treatment plan includes an event for setting up a patient journal, and method further comprises automatically setting up the patient journal at the date specified by said event.

In one form, the event for setting up the patient journal is pre-specified by the treatment plan template on which the finalized treatment plan is based.

In one form, the event for setting up the patient journal is added to the finalized treatment plan by the control module in response to an analysis conducted on the further information about the patient.

Preferably, the method further comprises automatically analysing inputs into the patient journal according to one or more analysis parameters.

### BRIEF DESCRIPTION OF THE DRAWINGS

In order that the present invention can be more readily understood, reference will now be made to the accompanying drawings which illustrate preferred embodiments of the invention and wherein:

FIG. 1 illustrates a healthcare system and a control module according to the present invention;

FIGS. 2A and 2B illustrate a general workflow and operation of the healthcare system to generate a treatment plan template;

FIGS. 3 and 4 illustrate a general workflow and operation of the healthcare system to generate a treatment plan;

FIGS. 5A and 5B illustrate a general workflow and operation of the healthcare system to optimize a treatment plan implementation calendar;

FIGS. 6A and 6B illustrate a general workflow and operation of the healthcare system to automatically revise a treatment plan implementation calendar;

FIG. 7 illustrates the healthcare system and a messaging module according to the present invention;

FIG. 8 illustrates an operation of the healthcare system for generating and completing a patient journal;

FIG. 9 illustrates a portal map of a Specialist and GP portal;

FIG. 10 illustrates a Dashboard page of the Specialist and GP portals;

FIGS. 11A to 110 illustrate a Patient Listing page, Add Patient page and Patient Profile page of the Specialist and GP portals;

FIG. 12 illustrates a Test Listing page of the Specialist and GP portals;

FIG. 13A illustrates a Treatment Plan Template Listing page of the Specialist and GP portals;

FIGS. 13B to 13F illustrate an Add Treatment Plan Template page, Add Event page, Treatment Plan display page, and Treatment Plan Listing page of the Specialist and GP portals;

FIGS. 14A and 14B illustrate a Questionnaire page of the Specialist and GP portals;

FIGS. 15A and 15B illustrate a Task list page and Calendar display page of the Specialist and GP portals;

FIGS. 16A and 16B illustrate a Message list page and chat page of the Specialist and GP portals;

FIG. 17 illustrates a portal map of a Nurse portal;

FIG. 18 illustrates a Dashboard page of the Nurse portal;

FIG. 19 illustrates a Treatment plan listing page of the Nurse portal;

FIGS. 20A and 20B illustrate the Task List page and Calendar display page of the Nurse portal;

FIG. 21 illustrates a portal map of the Patient portal;

FIG. 22 illustrates a Dashboard page of the Patient portal; and

FIG. 23 illustrates a Treatment plan page of the Patient portal.

### DETAILED DESCRIPTION OF PREFERRED EMBODIMENTS

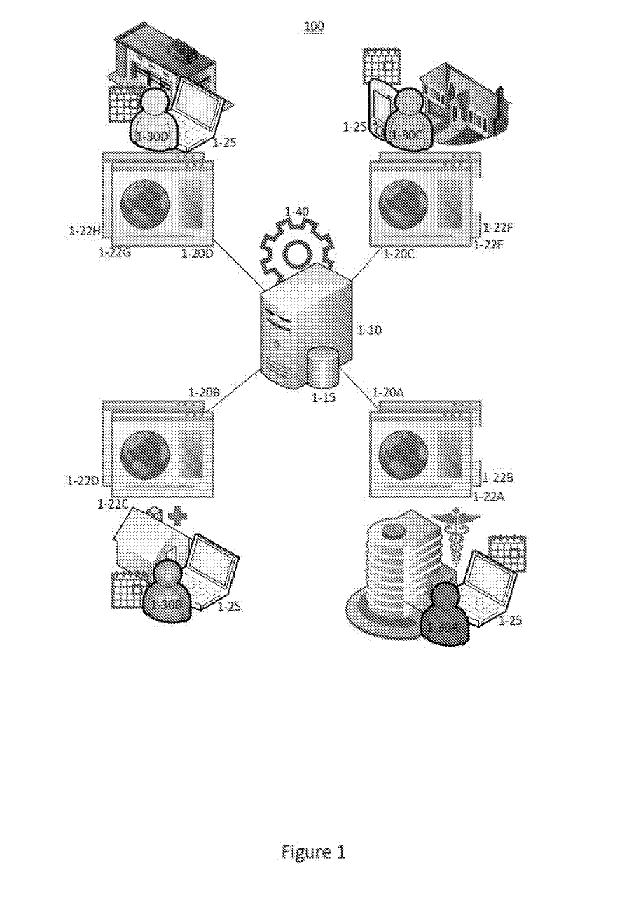

A healthcare system 100 according to the present invention is illustrated in FIG. 1. The healthcare system 100 comprises a central server 1-10 and database 1-15. Whilst illustrated in FIG. 1 as a single computing device and a single database, it is to be understood that the central server 1-10 may have a distributed arrangement and the database 1-15 may similarly have a distributed arrangement.

The healthcare system 100 further comprises a plurality of portal sets 1-20A, 1-20B, 1-20C, 1-20D (collectively referred to as portal sets 1-20), which provide remote network access to the central server 1-10 and database 1-15. The portal sets 1-20 include, but are not limited to, Specialist portal set 1-20A, GP portal set 1-20B, Patient portal set 1-20C, and Professional portal set 1-20D. Each portal set 1-20 comprises one or more portals 1-22A, 1-22B, 1-22C, 1-22D, 1-22E, 1-22F, 1-22G, 1-22H (collectively referred to as portals 1-22) specific to the needs of the users of the portals 1-22. The portals 1-22 are accessible by the electronic devices 1-25 operated by users 1-30A, 1-30B, 1-30C, 1-30D (collectively referred to as users 1-30) of the healthcare system 100. In a preferred embodiment, the electronic devices 1-25 access the portals 1-22 via a dedicated application running on each electronic device 1-25. However, the portals 1-22 may also be accessed through a web browser running on each electronic device 1-25.

The users 1-30 of the healthcare system 100 include a Specialist Doctor and/or a Specialist Nurse 1-30A (hereinafter referred to respectively as the “Specialist” and the “Specialist Nurse”), a GP and/or GP Nurse 1-30B (hereinafter referred to respectively as the “GP” and “GP Nurse”), a Patient and/or Patient's Career 1-30C, and other Healthcare Professional 1-30D (e.g. pathologist, radiologist, other Specialist Doctor, physiotherapist, laboratory technicians, allied health practitioners, and the like). Together, the users 1-30 comprise a care unit for the Patient 1-30C receiving healthcare via the healthcare system 100.

Logically, the Specialist and Specialist Nurse comprise a Specialist Team, the GP and GP Nurse comprise a GP Team, the Patient and Carer comprises a Patient Team, and the other Healthcare Professionals comprises a Professionals Team. For conciseness and ease of description, users of a same team will be referred to hereinafter with the same reference number. Specifically, the Specialist and Specialist Nurse will be referred to with the same reference number 1-30A, the GP and GP Nurse will be referred to with the same reference number 1-30B, the Patient and the Patient's carer will be referred to with the same reference number 1-30C, and the other Healthcare Professionals involved in the provision of healthcare to the Patient 1-30C will be referred to with reference number 1-30D.

In a preferred embodiment, the Specialist portal set 1-20A includes a portal 1-22A designed for the specific use of the Specialist 1-30A, and a further portal 1-22B designed for the specific use of the Specialist Nurse 1-30A. Similarly, in the preferred embodiment, the GP portal set 1-20B includes a portal 1-22C designed for the specific use of the GP 1-30B, and a further portal 1-22D designed for the specific use of the GP Nurse 1-30B. Similarly, in the preferred embodiment, the Patient portal set 1-20C includes a portal 1-22E designed for the specific use of the Patient 1-30C, and a further portal 1-22F designed for the specific use of the Patient's Carer 1-30C. Similarly, in the preferred embodiment, the Professional portal set 1-20B includes specific portals 1-22G each designed for the specific use of the various Healthcare Professionals 1-30D who may be involved in the provision of healthcare to the Patient of 1-30C. The Professional portal set 1-20B may additionally (or alternatively) include a general portal 1-22H that is designed for the general use of any healthcare professionals.

As will be described in greater detail below, the Specialist portal 1-22A provides one or more of the following functionality, but is not limited to only the following functionality:
Creation and recordation of treatment plan templates in the central server 1-10 and database 1-15;
Creation of question sets and answer sets, each linked to a treatment plan template, in the central server 1-10 and database 1-15;
Creation of draft treatment plans;
Review and revision of draft treatment plans submitted to the Specialist by other members of the care unit;
Digital sign-off and finalization of draft treatment plans;
Dissemination of finalized treatment plans to the care unit;
Display of treatment plan implementation calendar specific to Specialist, which implementation calendar includes, but is not limited to, one or more of: task lists, timelines, milestones, reminders, appointments, automatically generated draft or final medication prescription, and automatically generated draft or final test requests;
Creation of search parameters for the central server 1-10 to search for test results in the database 1-15 and 3^{rd} party databases;
Creation of analysis parameters for the central server 1-10 to analyse test results and other information;
Display of all treatment plans for all patients belonging to a care unit of which the Specialist is a member; and/or
Secured messaging between members of each care unit the Specialist is a member of;

As will be described in greater detail below, the Specialist Nurse portal 1-22B provides one or more of the following functionality, but is not limited to only the following functionality:
Creation of draft treatment plan assisted by answer sets provided in response to a question set;
Restricted revision of draft and final treatment plans for diseases and conditions requiring Specialist oversight and authorization;
Dissemination of finalized treatment plans to the care unit
Display of treatment plan implementation calendar specific to the Specialist Nurse, which implementation calendar includes, but is not limited to, one or more of: task lists, timelines, milestones, reminders, appointments, automatically generated draft or final medication prescriptions, and automatically generated draft or final test requests;
Restricted revision of draft and final treatment plans for diseases and conditions requiring Specialist oversight and authorization;
Creation of general, specific, and guided patient logs/journals for completion by the Patient 1-30C, which logs/journals are in a format suitable for analysis by the server using prescribed analysis parameters;
Creation of search parameters for the central server 1-10 to search for test results in the database 1-15 and 3^{rd} party databases;
Creation of analysis parameters for the central server 1-10 to analyse test results and other information;
Notification and review of automated alerts generated by the search parameters or analysis parameters;
Display of all treatment plans for all patients belonging to a care unit of which the Specialist is a member; and/or
Secured messaging/communication between members of the care unit.

As will be described in greater detail below, the GP portal 1-22C and GP Nurse portal 1-22D provide one or more of the following functionality, but is not limited to only the following functionality:
Creation of draft treatment plan for specialist-treated diseases/conditions assisted by answer sets provided in response to a question set;
Restricted revision of draft and final treatment plans for specialist-treated diseases/conditions;
Creation, dissemination, and unrestricted revision of draft and final treatment plans for conditions not requiring Specialist oversight and authorization;
Submission of draft treatment plans to Specialist for authorization;
Display of treatment plan implementation calendar specific to the GP, which implementation calendar includes, but is not limited to, one or more of: task lists, timelines, milestones, reminders, appointments, automatically generated draft of final medication prescriptions, and automatically generated draft or final test requests;
Creation of search parameters for automatic searching, by the central server, of test results in the databases of test centres;
Creation of analysis parameters for automatic analysis, by the central server, of test results;
Creation of general, specific, and guided patient logs/journals for completion by the patient, which logs/journals are in a format suitable for analysis by the server using prescribed analysis parameters; and/or
Display of all treatment plans for all patients belonging to a care unit of which the GP is a member;
Secured messaging/communication between members of the care unit.

As will be described in greater detail below, the Patient portal 1-22E and Carer portal 1-22F provides one or more of the following functionality, but is not limited to only the following functionality:
Receiving and sending messages from/to members of the care unit;
Display of treatment plan implementation calendar specific to the Patient, which management plan includes task lists, timelines, milestones, reminders, and appointments;
Completion of general, specific, and guided patient journals/logs to track patient condition, statistics, and other information; and/or
Creation and completion of ad hoc patient journal/log to track and record information deemed relevant by the patient;
List of Specialist and GP diagnosed medical conditions and approved medications.

In addition to the above, the healthcare system 100 further includes a control module 1-40. The control module 1-40 is configured and adapted to control and manipulate an operation of the central server 1-10 and/or database 1-15. In a preferred embodiment, the control module 1-40 is realized as software code and/or instructions configured to configure and control the central server 1-10 and/or database 1-15. In another embodiment, the control module 1-40 is realized as a combination of software code and hardware, including logic gates, transistors, memory units, and processing units, and may form and/or include a part of the central server 1-10 and/or database 1-15. In a further embodiment, the control module 1-40 is realized as hardware, comprising transistors and other micro-electronic circuitry.

As will be described in greater detail below, the central server 1-10 and database 1-15 when configured and controlled by the control module 1-40, or when comprising part of the control module 1-40, is operable to:
Analyse and process treatment plans to automatically generate treatment plan implementation calendars specific to each member of the care unit;
Generate notifications, alerts, reminders, and automated messages for and to send to members of the care unit, in accordance with the treatment plan;
Analyse patient logs/journals in accordance with prescribed analysis parameters;
Generate notifications, alerts, triggers, and changes to the treatment plan when patient logs/journal entries satisfying one or more analysis parameters are identified;
Search the database 1-15 and/or 3^{rd} party databases for test results matching prescribed search parameters;
Analyse test results in accordance with prescribe analysis parameters;
Generate notifications, alerts, triggers, and changes to the treatment plan when test results satisfying one or more analysis parameters are identified;
Generate test requests (e.g. blood tests, radiology tests) in a dynamic manner; and/or
Generate digital prescriptions.

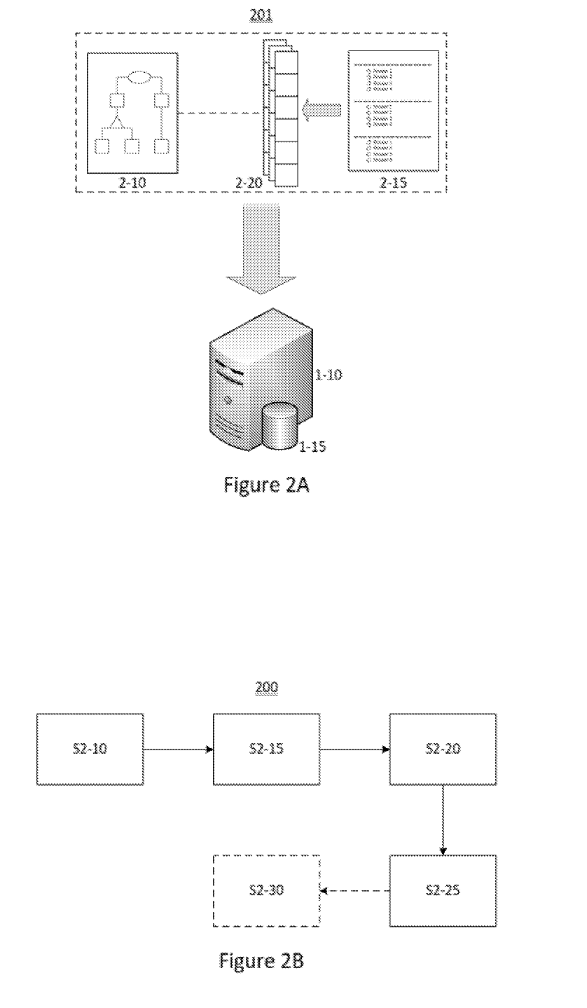

Referring now to FIGS. 2A and 2B, a general workflow 201 and operation 200 of the healthcare system 100, and specifically of the central server 1-10, database 1-15, control module 1-40 and Specialist portal 1-22A, to generate and make available for use a treatment plan template 2-10 is described.

As illustrated by the general workflow 201, a treatment plan template 2-10 is created by the Specialist 1-30A by way of the Specialist portal 1-22A (or by the GP 1-30B by way of the GP portal 1-22 C) to comprise a specific arrangement and structure of events suited for treating a specific health condition (e.g. a specialist-treated chronic disease or GP-treated health condition) given certain characteristics (e.g. symptoms, age, gender, complications, etc.) of a patient. The treatment plan 2-10 includes, but is not limited to, the specifying of:
Drugs to be taken (what, when, how much)
Information regarding drugs as reference
Tests to be performed (what, when, why, window of acceptability)
Electronic test request forms
Follow-ups to be made (who, what, when)
Reports to be submitted (who, what, when)
Trigger conditions and corresponding contingent actions

The treatment plan template 2-10 is stored in and accessible from the central server 1-10 and/or database 1-15.

In addition to the treatment plan template 2-10, a question set 2-15 is created. The question set 2-15 comprises a plurality of questions created by the Specialist 1-30A that are pertinent to the Patient 1-30C and the treatment of the Patient's disease/condition.

In the preferred embodiment, the question set 2-15 is structured such that the majority of answers to the questions are structured data input fields (e.g. a multiple choice answers) and therefore generated a finite combination of answers. Each combination of answers is hereinafter referred to as an answer set 2-20. The various combinations of answer sets 2-20 are stored in the central server 1-10 and/or database 1-15, and each treatment plan template 2-10 is associated with at least one answer set 2-20. The provision of the question set 2-15 containing questions created by the Specialist 1-30A facilitates the treatment of the Patient 1-30C by non-Specialist, for example the GP 1-30B, by ensuring that information necessary for and pertinent to the treatment of the Patient 1-30C is obtained, which information then informs the generation of an appropriate treatment plan, as will be described below.

In the above manner, an answer set 2-20 is generated each time the question set 2-15 is answered, which then identifies at least one treatment plan template 2-10, namely the treatment plan template(s) 2-10 associated with the specific answer set 2-20.

FIG. 2B illustrates the operation 200 for generating and making available a treatment plan template 2-10.

At S2-10, a treatment plan template 2-10 for the condition, and specific to a given set of patient characteristics, is created by the Specialist 1-30A via the Specialist portal 1-22A. The treatment plan template is stored in the central server 1-10 and/or database 1-15.

At S2-15, the Specialist 1-30A, via the Specialist portal 1-22A, creates a question set 2-15 that is specific to the condition being treated. The question set 2-15 includes questions that are relevant to the treatment of the disease/condition and the patient. In an exemplary embodiment where the condition being treated is, for example, Hepatitis C, relevant questions may include:
Degree fibrosis of the liver;
The genotype of the virus; and
Whether previous treatment(s) was attempted

Once created, the question set 2-15 is stored in the central server 1-10 and/or database 1-15. If a question set 2-15 for the condition has previously already been created, creation of a question set 2-15 may be skipped.

At S2-20, an answer set 2-20, comprising of a combination of answers to the question set 2-15 that best matches the patient characteristics and condition for which the treatment plan template 2-10 is being created, is determined and selected.

At S2-25, an association between the selected answer set 2-20 and the treatment plan template 2-10 is generated by the central server 1-10 and stored in the central server 1-10 and/or database 1-15.

In a preferred embodiment, an additional step S2-30 allows a treatment plan template 2-10 to be published and/or unpublished. Once published, a treatment plan template is available for use by other members of a care unit to create a draft treatment plan (described in greater detail below).

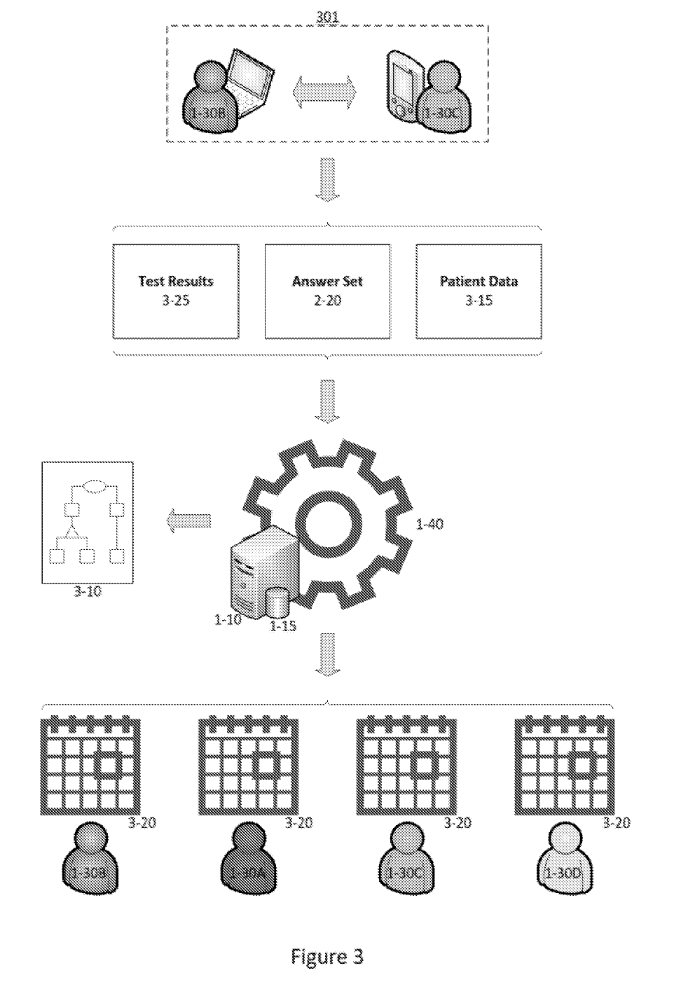

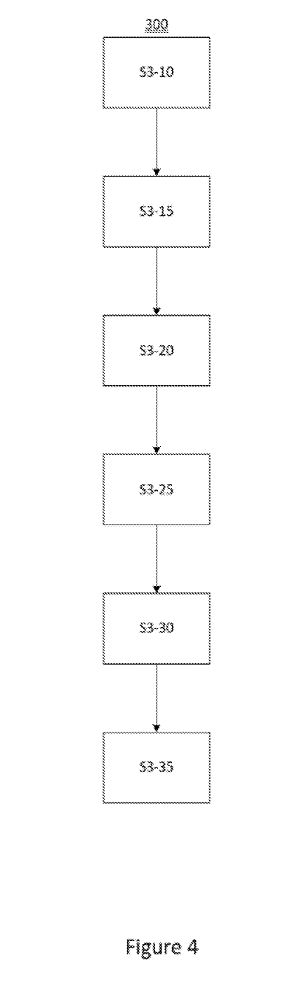

Referring now to FIGS. 3 and 4, a general workflow 301 and operation 300 of the healthcare system 100, and specifically the central server 1-10, database 1-15, control module 1-40 and GP portal 1-22C, to generate a treatment plan 3-10 and associated treatment plan implementation calendars 3-20 is described.

It is to be understood that while the operation 300 is described with reference to the GP portal 1-22C (operated by the GP 1-30B) as the portal facilitating the generating of the draft treatment plan, the healthcare system 100 is not so limited. Rather, generation of the draft treatment plan is possible through any portal 1-22 that has been configured to permit such action, including for example the Specialist portal 1-22A, Specialist Nurse portal 1-22B and GP Nurse portal 1-22D, under the operation of the appropriate user of those portals 1-22.

With reference to the general workflow 301 illustrated in FIG. 3, in order to generate a draft treatment plan a number of inputs are first collected and subsequently inputted to the control module 1-40. The inputs to be collected and inputted to the control module 1-40 include, but are not limited to, one or more of:
An answer set 2-20;
Patient data 3-15; and
Patient test results 3-25.

The inputted answer set 2-20 is an answer set 2-20 obtained through completion of the question set 2-15 by the GP 1-30B via the GP portal 1-22C. The patient data 3-15 includes data obtained through interrogation of the Patient 1-30C, whether manually a member 1-30 of the care unit or automatically by health sensors such as heart rate monitors, glucose monitors, pacemakers, hearing aids, and any other attached, implanted, or injected sensor. The test results 3-25 include results obtained from laboratory tests (e.g. blood tests, radiology scans, ultrasound scans, urine tests, etc.), and test results 3-25 obtained from the Patient 1-30C, for example those entered via the Patient portal 1-22E (as will be described in greater detail below). The test results 3-25 obtained from laboratory tests may be obtained from the database 1-15, and/or from 3^{rd} party databases of the testing laboratories, and/or entered manually by a member of the Patient's care unit.

The control module 1-40, once provided with one or more of the above information, and in particular the answer set 2-20, selects a treatment plan template 2-10 from which to generate a draft treatment plan. Additionally, the control module 1-40 may modify the draft treatment plan in view of one or more of the above pieces of information. One modified as necessary, the control module 1-40 transmits the draft treatment plan to the GP 1-30B via the GP portal 1-22C for an initial review. As will be described below in greater detail with reference to FIG. 4, the control module 1-40 is further configured to effect revisions to the draft treatment plan by one or more members of the care unit, including the GP 1-30B, Specialist 1-30A, and Specialist Nurse 1-30A, and to ultimately generate a finalized treatment plan 3-10 that has been digitally authorized by the Specialist 1-30A.

The control module 1-40, upon generation of the finalized treatment plan 3-10, generates customized treatment plan implementation calendars 3-20 for each member of the Patient's care unit. Specifically, each treatment plan implementation calendar 3-20 is generated to comprise reminders, events, appointments, meetings, and general notes that are specific to and relevant for each member of the Patient's care unit.

A treatment plan implementation calendar 3-20 for the Patient 1-30C may, for example, include:
Dates/timings for drug doses;
Information regarding drugs
Dates/timings/locations for tests;
Electronic test request forms
Dates/timings/locations for GP or Specialist visits/appointments;
Dates/timings/content for submission of Patient journal entries.
Dates/timings for receipt and execution of medication prescription scripts and/or test requests;

Conversely, a treatment plan implementation calendar 3-20 for the Specialist Nurse 1-30A or GP Nurse 1-30B may, for example, include:
Dates/timings to check in/follow up on Patient;
Dates/timings of various reports due from Specialist/GP;
Dates/timings of various reports due to Specialist/GP;
Dates/timings/locations of Patient visits;
Dates/timings of test results due from Patient.
Dates/timings for issuance of medication prescription scripts and/or test requests;
Draft prescription scripts (electronic and/or for hard copy print out) and test requests (electronic and/or for hard copy print out) completed with information derived from the treatment plan, for example:
   Drug/Test
   Drug dosages/Special Test Requirements
   Repeats
   Patient name
   Date
   Specialist's/GP's prescriber registration number

While, a treatment plan implementation calendar 3-20 for the Specialist 1-30A or GP 1-30B may, for example, include:
Dates/timings to check in/follow up on Patient;
Dates/timings of various reports due from other members of the care unit;
Dates/timings of various reports due to other members of the care unit;
Dates/timings/locations of Patient visits/appointments;
Dates/timings of test results due from Patient;
Dates/timings for issuance of medication prescription scripts and/or test requests;
Draft prescription scripts (electronic and/or for hard copy print out) and test requests (electronic and/or for hard copy print out), partially completed with information derived from the treatment plan, for example:
   Drug/Test
   Drug dosages/Special Test Requirements
   Repeats
   Patient name
   Date
   Specialist's/GP's prescriber registration number

In the preferred embodiment, each treatment plan implementation calendar 3-20 is generated in electronic form and stored on, or otherwise made accessible and available to, respective electronic devices 1-25 of each member of the care unit. Further, in the preferred embodiment, each calendar 3-20 is provided with a set of permission settings controlling which other members of the care unit, if any, can view a given calendar 3-20.

FIG. 4 illustrates the steps of the operation 300 for generating the treatment plan 3-10 and treatment plan implementation calendars 3-20.

At S3-10, one or more of an answer set 2-20, patient data 3-15, and patient test results 3-25 are received by the control module 1-40. Collection of such information is effected by one or more of manual entry by a member of the care unit (e.g. the GP 1-30B) via a portal 1-22, automated sensing from health sensors, automated and/or user initiated searching of the database 1-15 or other 3^{rd} party database, and responding to a question set 2-15. Members of the care unit with sufficient rights (e.g. the Specialist 1-30A) may skip this step.

At S3-15, using the information received at S3-10, the control module 1-40 selects a treatment plan template 2-10, and modifies the selected treatment plan template 2-10 to produce a draft treatment plan.

At S3-20, the control module 1-40 transmits the draft treatment plan to appropriate members of the care unit, typically at least the member who initiated the creation of the draft treatment plan (e.g. GP 1-30B, GP Nurse 1-30B, Specialist Nurse 1-30A), for a first review. Manual modifications to the draft treatment plan may also be made at S3-20 by these members of the care unit. Preferably, the draft treatment plan is transmitted to the portals 1-22 of each appropriate member of the care unit, and thereby made viewable and modifiable.

At S3-25, the draft treatment plan is transmitted to the Specialist 1-30A, in particular to the Specialist portal 1-22A. Via the Specialist portal 1-22A, the Specialist 1-30A conducts a second review of the draft treatment plan. Further manual modifications to the draft treatment plan may be made at S3-25 by the Specialist 1-30A. The Specialist 1-30A, once all desired modifications have been made, authorizes the draft treatment plan to create a finalized treatment plan 3-10. In the preferred embodiment, the Specialist 1-30A authorizes the draft treatment plan by digitally signing the draft treatment plan.

At S3-30, the control module 1-40 analyses and processes the finalized treatment plan 3-10 to generate a plurality of treatment plan implementation calendars 3-20, one each for each member of the Patient's care unit as necessary. The control module 1-40 in particular generates each treatment plan implementation calendar 3-20 to comprise one or more of reminders, events, appointments, meetings, documents/attachments (e.g. medication prescriptions, test requests, other reference information), and/or general notes specific and relevant to each member of the Patient's care unit, based on information contained within the treatment plan 3-10 such as:
Task/event
Date
Location
Responsible parties
Subject parties
Medication
Dosages
Notes

In a preferred embodiment, included as part of the generation of the treatment plan implementation calendars 3-20 is the generation of medication prescriptions and test requests, which are then attached as documents with an appropriate treatment plan implementation calendar (e.g. the Patient's treatment plan implementation calendar) at an appropriate date (e.g. around the expected date when the previous allotment of medication runs out)

At S3-35, the plurality of treatment plan implementation calendars 3-20 are transmitted, or otherwise make accessible and available, to the electronic devices 1-25 of respective care unit members.

As previously mentioned, the general workflow 301 operation 300 described above has been described using an example where the GP 1-30B and GP portal 1-22C create a draft treatment plan for a specialist-treated disease, and which draft treatment plan therefore requires authorisation by the Specialist 1-30A. It is to be understood that the general workflow 301 and operation 300 is not so limited. Indeed, the general workflow 301 and operation 300 are applicable to other situations, including where:
The GP 1-30B and GP portal 1-22C are used to create a treatment plan for a GP-treatable disease, whereby the GP 1-30B can be the authorising entity;
The GP Nurse 1-30B and GP Nurse portal 1-22D are used to create a treatment plan for a GP-treatable disease, whereby the GP 1-30B can be the authorising entity;
The GP Nurse 1-30B and GP Nurse portal 1-22D are used to create a treatment plan for a Specialist-treatable disease, whereby the Specialist 1-30A is the authorising entity;
The GP Nurse 1-30B and GP Nurse portal 1-22D are used to create a treatment plan for a Nurse-supervisable condition, whereby the GP Nurse 1-30B or the GP 1-30B can be the authorising entity.

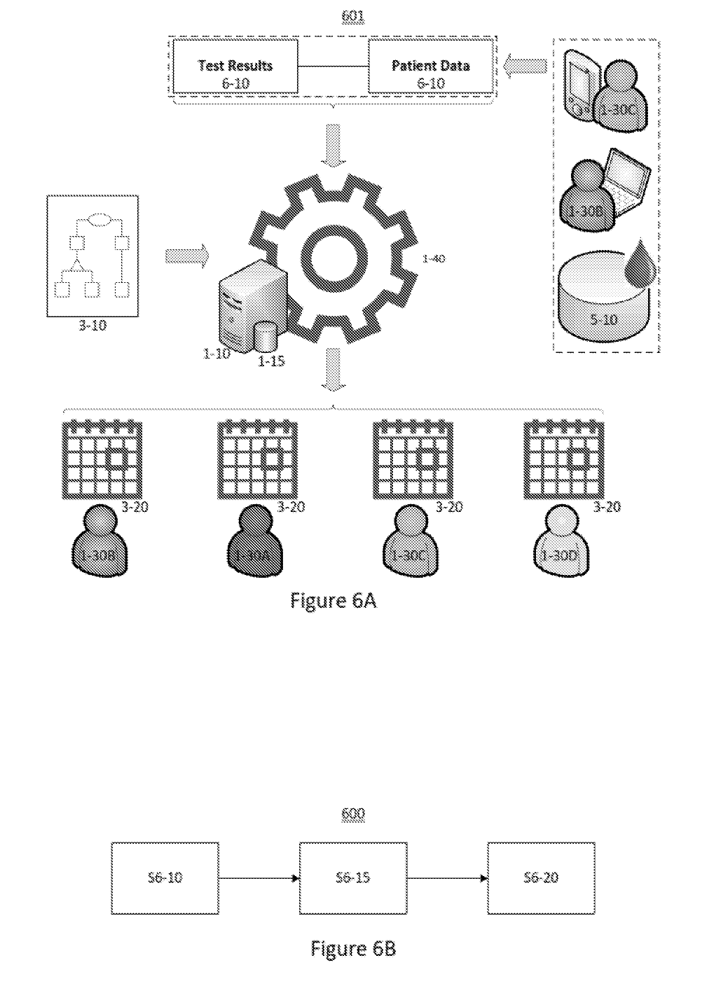

Changes to a finalized treatment plan 3-10 may be desired for any number of reasons including, for example, changes in a condition of the Patient 1-30C, or as will be described in greater detail below with reference to FIGS. 6A and 6B, the activation of a trigger configured to monitor and analyse a variety of information.

Changes to a finalized treatment plan 3-10 are generally changes that materially affect the overall plan or strategy for treating the Patient 1-30C. In contrast, changes such as changes to reschedule appointment dates, times, and locations, reporting deadlines, and the like, and which do not materially affect the overall treatment plan or strategy, should preferably not be effected by changing the finalized treatment plan 3-10 but rather by changing the treatment plan implementation calendars 3-20.

One example of a change in the Patient's condition that would necessitate a change to a finalized treatment plan 3-10 might be the development of an allergic reaction to the drug prescribed by the finalized treatment plan 3-10. Another example might be the onset of an illness (e.g. influenza, diarrhoea, fever, etc.) that needs to be treated before the Patient 1-30C can resume treatment according to the finalized treatment plan 3-10.

Changes to a finalized treatment plan 3-10 are effected by way of an operation largely similar to that of the above operation 300 for creating a treatment plan 3-10. Specifically, a draft change to the finalized treatment plan 3-10 is made (e.g. S3-20) by a member of the care unit via an appropriate portal 1-22, which draft change is then transmitted (e.g. S3-25) to the Specialist 1-30A for revision, if necessary, and authorization. Once authorized, the control module 1-40 analyses and processes (e.g. S3-30) the changed treatment plan 3-10 and revises the treatment plan implementation calendars 3-20.

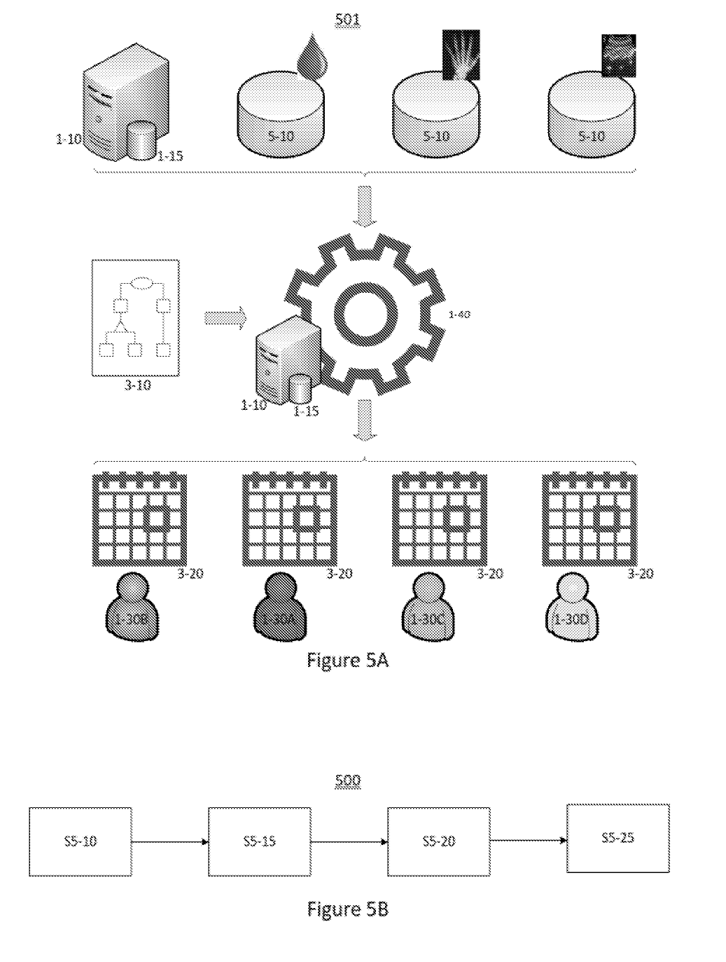

Changes to one or more treatment plan implementation calendars 3-20 may also be desired for any number of reasons including, for example, a change in availability of one or more members of the Patient's care unit, or, as will be described in greater detail below with reference to FIG. 5, a positive result from an automated search for suitable test results conducted by the control module 1-40, or as will also be described in greater detail below with reference to FIGS. 6A and 6B, the activation of a trigger configured to monitor and analyse a variety of information.

Changes to a treatment plan implementation calendar 3-20 may be made by any member of the care unit through operation of their portal 1-22. The control module 1-40 is operable to then automatically update the implementation calendars 3-20 of any other member of the care unit who would be affected by this change. Changes may be made as a proposed change and the proposed changes accepted ‘tentatively’ and/or responded to with alternative proposed changes, or can also be declined. Changes to a treatment plan may also be made or proposed automatically by the control module 1-40, for example in response to positive or negative results of searches (e.g. searches for test results) and analyses (e.g. analyses of test results) conducted thereby.

With reference now to FIGS. 5A and 5B, a general workflow 501 and operation 500 for optimizing a treatment plan implementation calendar 3-20 is described.

There are a variety of ways in which a treatment plan implementation calendar 3-20 can be optimized, including by scheduling events such as face-to-face reviews at a time where multiple issues can be addressed simultaneously, by ensuring that reviews are not conducted prior to the information required to be reviewed being available, and by reducing duplication. For example, a treatment plan 3-10 often includes one or more tests, such as blood tests. It is not uncommon for the same tests to have already been performed by another health provider for an unrelated condition. The conduct of such recent and prior tests obviates the need for the test to again be conducted if the results from the recent test can be obtained.

FIG. 5A illustrates the general workflow 501 for specifically optimizing treatment plan implementation calendars 3-20 by, wherever possible, avoiding multiple, redundant tests. To assist with the optimization process, tests (e.g. a blood test) are preferably specified in the treatment plan by a number of parameters including:
Test type (e.g. Hep C genotype)
Window of acceptability (e.g. conducted within 4 weeks prior to “Event 3”)

The control module 1-40 extracts from the treatment plan 3-10 the test type and window of acceptability for each test specified by the treatment plan, and determines a search commencement date and search end date. Together, the test type, search commencement date, search end date, and any other information deemed relevant and extracted or otherwise determined by the control module 1-40 form search parameters used by the control module 1-40 for the searching of appropriate test results or test requests. Alternatively, or additionally, search parameters may be manually set up and provided to the control module 1-40 by the Specialist 1-30A, Specialist Nurse 1-30A, GP 1-30B, GP Nurse 1-30B, or other Professional 1-30D through their respective portals 1-22.

At the commencement of the search commencement date, the control module 1-40 searches the database 1-15 and other 3^{rd} party databases 5-10, including those of testing laboratories, for test results or test requests satisfying the parameters. If appropriate test results are found, the results of the test are recorded in the database 1-15 and the test that was originally scheduled by the treatment plan 3-10 is cancelled or marked as unnecessary. Alternatively, if an appropriate test request (e.g. a test request specified by a different treatment plan for another disease or condition of the Patient 1-30C) is found, the future results of that test request are identified for future retrieval (or that test request is itself modified to have its results sent also to the care unit), and the test that was originally scheduled by the treatment plan 3-10 is marked as unnecessary. The originally scheduled test may be marked as unnecessary by, for example, updating the treatment plan implementation calendars 3-20 to remove the scheduled test or to have the scheduled test marked as completed.

The searching for appropriate test results and test requests is conducted periodically, for example on a daily basis, commencing from the search commencement date and ceasing at the search end date.

In the preferred embodiment, if by the search end date, an appropriate test result and/or test request is not found, the control module 1-40 generates a test request using information provided in or otherwise derivable from the treatment plan 3-10, such as test type required, patient name, requesting doctor's name, special requests, and the like. The generated test request can be sent to the Patient 1-30C via the Patient portal 1-22E of Carer portal 1-22F or directly to a test laboratory.

FIG. 5B illustrates the steps of the optimization operation 500 when applied to optimize the treatment plan for redundant tests.

At S5-10, the control module 1-40 analyses and processes the treatment plan 3-10 to extract therefrom information for each specified test, such as a window of acceptability and the type of each test, to generate search parameters. Alternatively, the search parameters are provided to the control module 1-40 by the Specialist 1-30A, Specialist Nurse 1-30A, GP 1-30B, or GP Nurse 1-30B via respective portals 1-22. Two search parameters which are either extracted from the treatment plan 3-10 (in particular, the window of acceptability) or provided by a member of the care unit, are a search commencement and a search end date.

At S5-15, the control module 1-40 commences, at the search commencement date, a search for test results or test requests that satisfy the search parameters. This search is performed periodically until an appropriate test result or test request is found, or the arrival of the search end date.

At S5-20, if an appropriate test result or test request is found, the control module 1-40 updates all relevant treatment plan implementation calendars 3-20 to remove the test from these calendars 3-20, or otherwise mark the test as completed. In the case where an appropriate test request is found, the control module 1-40 further identifies the test request as one whose results should also be provided to the care unit.

At S5-25, if no appropriate test result or test request is found by the search end date, the control module 1-40 generates a test request using information provided in or otherwise derivable from the treatment plan 3-10, such as test type required, patient name, requesting doctor's name, special requests, and the like. The generated test request is then sent to the Patient 1-30C via the Patient portal 1-22E of Carer portal 1-22F, to another member of the care unit, and/or directly to a test laboratory.

The generated test request can be sent to the Patient 1-30C via the Patient portal 1-22E of Carer portal 1-22F or directly to a test laboratory.

The example illustrated in FIGS. 5A and 5B represents just one manner in which the treatment plan implementation calendars 3-20 can be optimized, and the healthcare system 100 is not so limited.

In general terms, each event in a treatment plan implementation calendar 3-20 can be assigned with (or used to derive) one or more rules, such as:
Conduct no earlier than X<time unit> prior to Event Y
Conduct no earlier than X<time unit> after Event Y
Conduct no later than X<time unit> after Event Y
Conduct only if X is complete/available
Cancel if X obtained

While not described in detail, but derivable from the general work flow 501 and operation 500, further examples in which the treatment plan implementation calendars 3-20 can be optimized by way of the above rules include:
Ensuring that certain appointments scheduled for the specific purpose of reviewing a certain matter are scheduled only after sufficient information to perform the review is available, and rescheduling the appointment otherwise; and
Amalgamating blood drawn appointments (from different treatment plans).

A general form of operation 500 can therefore be described as comprising a step (S5-10) of analysing and processing the treatment plan 3-10 to extract details about an event, a step (S5-15) of deriving or otherwise obtaining from the extracted details a set of rules governing the event, a step (S5-20) of searching for conditions matching the governing rules, and a step (S5-25) of updating the treatment plan implementation calendars 3-20 appropriately in the event a matching condition is found.

With reference to FIGS. 6A and 6B, a general workflow 601 and operation 600 for automatically changing/revising a treatment plan implementation calendar 3-20 (and/or treatment plan 3-10) is described. Whilst the following description of the general workflow 601 and operation 600 is describe in respect to the revising of a treatment plan implementation calendar 3-20, it is to be understood the general workflow 601 and operation 600 are equally applicable to the revision of the treatment plan 3-10.

In one embodiment of the present invention, as illustrated by the general workflow 601 of FIG. 6A, a treatment plan implementation calendar 3-20 is automatically revised in response to triggers caused by analysing information 6-10 input to or otherwise obtained by the control module 1-40. The revision (hereinafter referred to as a triggered revision) to the treatment plan implementation calendar 3-20 may be specific to each trigger, or may be a default action, as will be described in greater detail below.

The information input to or otherwise obtained by the control module 1-40 includes, but is not limited to, test results (e.g. blood tests, radiology tests, etc.), sensor data from the aforementioned health sensors (e.g. heart rate monitors, glucose monitors, pacemakers, hearing aids, etc.), and patient condition and statistics such as those entered via the Patient portal 1-22E and/or GP portal 1-22C.

The aforementioned triggers may be included as part of the treatment plan 3-10, derived therefrom, and/or entered manually after creation of the treatment plan 3-10 by the Specialist 1-30A, Specialist Nurse 1-30A, GP 1-30B, GP Nurse 1-30B, or healthcare Professional 1-30D via the appropriate portal 1-22. The triggers may also be derived from the aforementioned analysis parameters used to analyse test results, which may similarly be included as part of the treatment plan 3-10 and/or entered manually after creation of the treatment plan 3-10 by the Specialist 1-30A, Specialist Nurse 1-30A, GP 1-30B, GP Nurse 1-30B, or healthcare Professional 1-30D via the appropriate portal 1-22. The triggers include, for example:
Minimum/maximum heart rate;
Minimum/maximum blood pressure;
Minimum/maximum glucose measurement;
Minimum/maximum body temperate;
Minimum/maximum stool frequency; and
Minimum or maximum result from validated tools (e.g. a set of validated questions or tests used to assess a certain chronic illness).

The triggered revisions may be predetermined and specified as part of the treatment plan 3-10, specified during or after the creation of the triggers, or may be a default triggered revision, such as a revision to add to an appropriate treatment plan implementation calendar 3-20 a notification for a member of the care unit to contact the Patient 1-30C. As will be described later below, the triggered revision may alternatively be the sending of an electronic message to an appropriate member of the care unit.

The information 6-10 input to or otherwise obtained by the control module 1-40 may be input/obtained periodically by the control module 1-40 by periodically searching for such information, and/or on a “push” basis, for example anytime a new test result is entered into the database 1-15, anytime a new test results is found by the control module 1-40 (for example, during the optimization operation 500), anytime a new report is obtained from the aforementioned health sensors, anytime a new patient log/journal is entered via the Patient portal 1-22E, and the like.

Upon receipt of the information, the control module 1-40 analyses the information to determine if any of the trigger conditions are satisfied. If a trigger condition is satisfied, the control module 1-40 makes all triggered revisions corresponding to the trigger condition.

The operation 600 for automatically revising a treatment plan implantation calendar 3-20 is described with reference to FIG. 6B.

At S6-10, information 6-10 is obtained by the control module 1-40. The information obtained by the control module 1-40 is, for example, information obtained from the control module 1-40 searching for and finding test results (e.g. blood tests, radiology tests, etc.), the control module 1-40 detecting that new test results have been recorded in the database 1-15, sensor data from the aforementioned health sensors (e.g. heart rate monitors, glucose monitors, pacemakers, hearing aids, etc.) being sent to the control module 1-40 or stored in the database 1-15, and entry of patient condition and statistics such as those entered via the Patient portal 1-22E.

At S6-15, the control module 1-40 analyses the information to determine if the information satisfies any triggers.

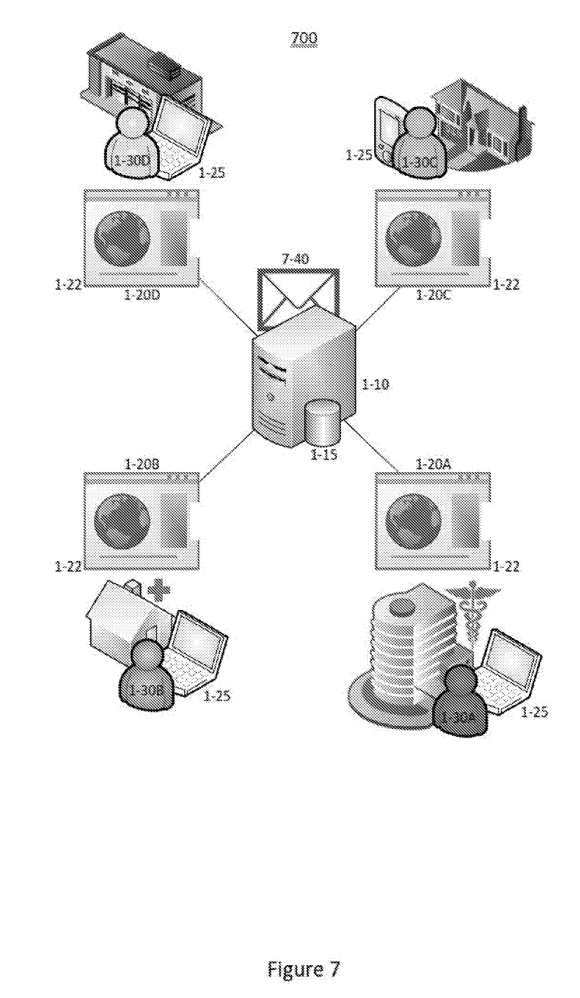

At S6-20, if a trigger is satisfied, the triggered revisions associated with the trigger is performed. If no triggered revisions are associated with the trigger, a general revision is performed. In one embodiment, the general revision is the addition of a new notification in appropriate treatment plan implementation calendars 3-20 notifying a respective member of the care unit to contact the Patient 1-30C. In another embodiment, instead of a general revision, a message-notification is sent to one or more members of the care unit via a messaging module 7-40 (FIG. 7) of the healthcare system 100.

With reference now to FIG. 7, the messaging module 7-40 of the healthcare system 100 according to the present invention is described. The messaging module 7-40 is configured and adapted to control and manipulate an operation of the central server 1-10 and/or database 1-15. In a preferred embodiment, the messaging module 7-40 is realized as software code and instructions configured to configure and control the central server 1-10 and/or database 1-15. In another embodiment, the messaging module 7-40 is realized as a combination of software code and hardware, including logic gates, transistors, memory units, and processing units, and may form and/or include a part of the central server 1-10 and/or database 1-15. In a further embodiment, the messaging module 7-40 is realized as hardware, comprising transistors and other micro-electronic circuitry.

The messaging module 7-40 configures and controls the central server 1-10 and database 1-15 to effect secure messaging between and amongst members of the Patient's care unit via respective portals 1-22.

With the messaging module 7-40 enabled, the aforementioned operation 600 may be modified such that step S6-20, as an alternative or in addition to performing the triggered revision, generates and sends a message to one or more members of the care unit.

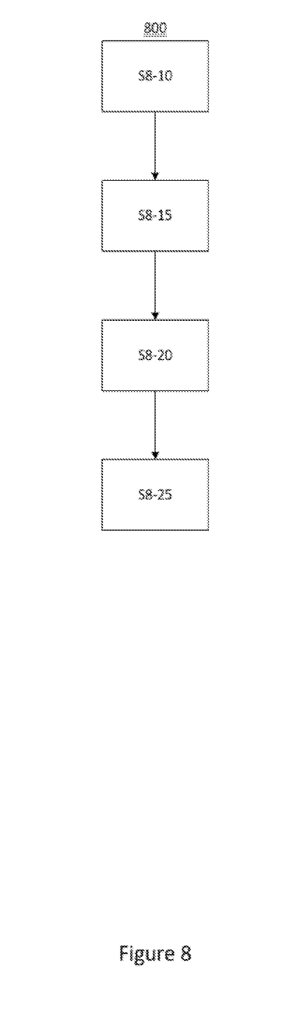

With reference now to FIG. 8, an operation 800 for generating and completing a patient journal is described. A patient journal is an electronic form that is configured for interaction with via the Patient portal 1-22E. Preferably, the patient journal is customized specifically for interaction via a mobile electronic device such as a smart phone or tablet.

In the preferred embodiment, the patient journal comprises a plurality of input fields, at least one of which is a structured data input field. As used herein, a structured data input field is an input field that allows entry of one or more fixed and predetermined inputs. A multiple choice input field is one example of a structured data input field.

The patient journal allows for the general and specific monitoring of the Patient 1-30C. Typically, general monitoring of the Patient 1-30C is effected by way of a general patient journal, which comprises a combination of mandatory input fields and optional or conditional input fields.

An exemplary general patient journal may include one or more of the following mandatory fields:
Date
Body temperature
Resting heart rate
Weight
Self-assessment of energy level (scale of 1-10)

The same exemplary general patient journal may then include the following optional or conditional input fields that can be added as necessary to a journal entry by the Patient 1-30C:
Sore throat
   Discomfort level (scale 1-10)
   Number of days
Fever
   Temperature
   Number of days
Diarrhoea
   Bowel movements per day
   Number of days
Chest pain
   Location (left/right)
   Discomfort level (scale 1-10)
   Number of occurrences
   Number of days

Specific patient journals for monitoring known conditions of the Patient 1-30C may also be set up. For example, a first specific patient journal may be set up to monitor the condition being treated by the treatment plan 3-10, and a second specific patient journal may be set up to monitor another known condition (e.g. coronary hearty disease) of the Patient 1-30C. The first and second specific patient journals differ from each other in that each comprise input fields that are specifically relevant to their respective conditions.

The need for a specific patient journal may be identified during creation of the treatment plan 3-10, and/or may arise from monitoring the general patient journal. A specific patient journal to monitor a Patient's diarrhoeic condition may be set up after, for example, analysis of the general patient journal suggests that the Patient 1-30C is currently experiencing diarrhoea and the diarrhoea is negatively affecting the Patient's chronic disease treatment (e.g. due to medication being passed before the body can absorb it).

A specific patient journal for monitoring, for example, the progress of the Patient's diarrhoeic condition may include the following exemplary input fields:
Date
Number of days since onset of diarrhoeic condition
Number of bowel movements per day
Stool condition
Urine colouration

Periodic entries of the above information by the Patient into the patient journal allows members of the care unit to monitor the Patient's diarrhoeic condition and determine what, if any, changes to the treatment plan 3-10 or treatment plan implementation calendar 3-10 may be necessary.

Both the general and specific patient journals may be, and indeed are preferably, customized to the Patient 1-30C. A Patient 1-30C with several known, existing, and ongoing conditions in addition to the disease being treated by the treatment plan 3-10 may, for example, have a general patient journal that requires more detail than even a specific patient journal of another Patient 1-30C who has no existing and ongoing conditions. In the preferred embodiment, the necessity of any journals required of the Patient 1-30C may be pre-specified as part of the treatment plan 3-10. The setup of such pre-specified patient journals may be pre-specified in a treatment plan template, and conducted automatically by the control module 1-40, for example, at S3-30 of operation 300 when the finalized treatment plan 3-10 is processed by the control module 1-40 to generate treatment plan implementation calendars 3-20, or at a later date as specified by the treatment plan 3-10, or as a triggered revision responsive to the aforementioned search and/or analysis parameters. Set up and customization of the patient journals may alternatively be conducted by the Specialist 1-30A, Specialist Nurse 1-30A, GP 1-30B, GP Nurse 1-30B, and/or healthcare professional 1-30D as deemed necessary.

At S8-10 of the operation 800, a patient journal is set up to include one or more input fields, each input field corresponding to a question or other request for information. The patient journal, once set up, is stored in the central server 1-10 and/or database 1-15.

At S8-15, the patient journal is made available to the Patient 1-30C, via the Patient portal 1-22E. Preferably, as part of the creation of the treatment plan 3-10 and/or treatment plan implementation calendars 3-20, reminders to the Patient 1-30C for making entries into the patient journal are included as are reminders to other appropriate members of the Patient's care unit to either check that the Patient 1-30C has made the required entries, and/or to review the entries. If such reminders were not included in the treatment plan 3-10 and/or treatment plan implementation calendars 3-20, they are then preferably included at S8-15. Analysis parameters may also be setup to check for timely journal entries by the Patient 1-30C, and to conducted an appropriate triggered revision or triggered message-notification if a journal entry is not timely entered.

At S8-20, the Patient 1-30C makes periodic entries into the patient journal as required. Preferably, with each periodic entry, an appropriate analysis parameter is applied by the control module 1-40 to the entry to determine if any triggered revision or other triggered response is necessary.

At S8-25, the entries are stored in the database 1-15. In the preferred embodiment, after each entry is stored in the database 1-15, the control module 1-40 analyses the entries for triggers, as previously described with reference to FIGS. 6A and 6B, and operation 600.

Whilst, as described above, in the preferred embodiment the patient journal comprises a plurality of input fields of which at least one is a structured data input field, the present invention is not so limited. The patient journal may also include unstructured data input fields (e.g. free text input), and one version of the patient journal may in fact by a Patient diary comprising predominantly, if not essentially entirely, of an unstructured data input field into which the Patient 1-30C may enter any information he/she may deem to be of relevance, whether to him/herself or to the care unit.

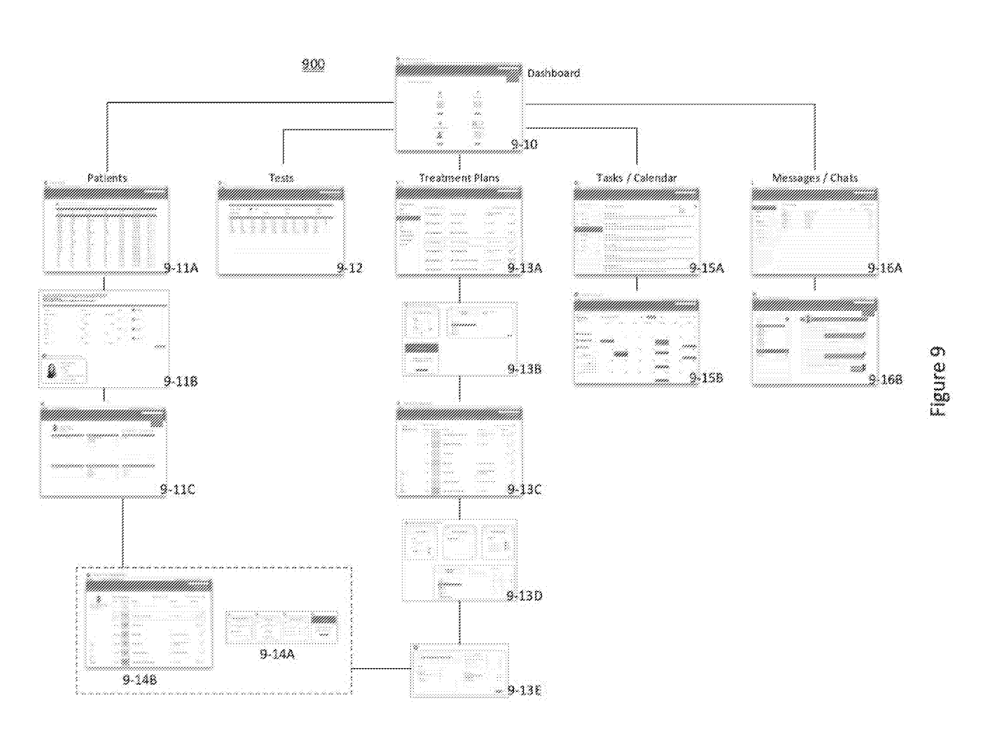

With reference now to FIGS. 9 to 16, the Specialist and GP portals 1-22A, 1-22C will be described in greater detail.

FIG. 9 illustrates a portal map 900 of the Specialist portal 1-22A. The GP portal 1-22C is similar to the Specialist portal 1-22A, and is likewise illustrated by the portal map 900. Accordingly, in the following description, descriptions in relation to the Specialist portal 1-22A and/or Specialist 1-30A should be understood as equally applicable to the GP portal 1-22C and GP 1-30B.

The portal map 900 sets out individual portal pages that make up each of the Specialist portal 1-22A and GP portal 1-22C. It is to be understood that the portal map 900 is not limited to only the portal pages illustrated in FIG. 9, and that other portal pages may be added to the portal map 900 or existing portal pages removed or replaced.

The Specialist portal 1-22A and GP portal 1-22C include the following portal pages:
Dashboard 9-10;
Patient Listing 9-11A;
Add Patient 9-11B;
Patient Profile 9-110;
Test Listing 9-12;
Treatment Plan Template Listing 9-13A;
Add Treatment Plan Template 9-13B;
Add Event 9-13C;
Treatment Plan Display 9-13D;
Edit Event 9-13E;
Treatment Plan Listing 9-13F
Questionnaire 9-14A;
Patient Treatment Plan 9-14B;
Task List 9-15A;
Calendar Display 9-15B;
Message List 9-16A; and
Chat 9-16B.

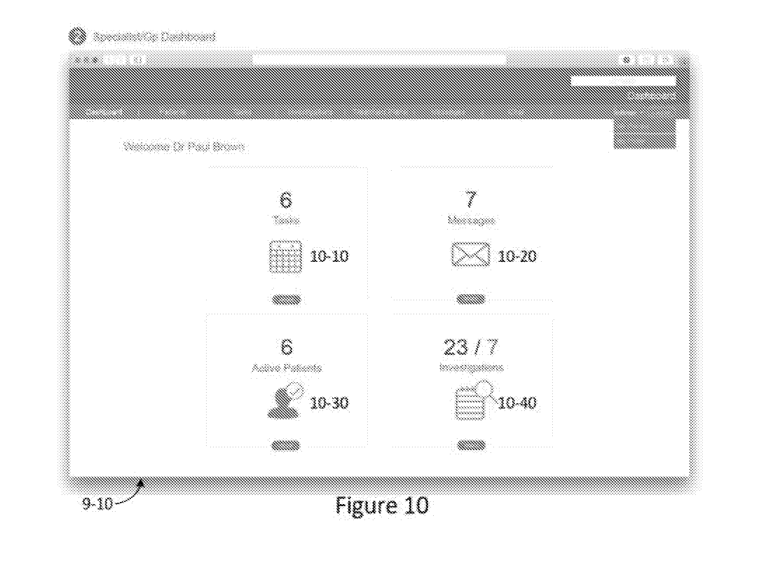

As illustrated in greater detail in FIG. 10, the Dashboard page 9-10 provides an overview of a Specialist's/GP's outstanding responsibilities, and includes a Task Summary 10-10, Messages Summary 10-20, Patient Summary 10-30, and a Test Summary 10-40.

The Task Summary 10-10 indicates a number of outstanding tasks still to be addressed by the Specialist/GP. Such tasks are set, for example, by the Specialist Nurse 1-30A (or equivalently, the GP Nurse 1-30B), the Specialist 1-30A (or equivalently the GP 1-30B), and/or the control module 1-40 for example by way of the generation of the treatment plan implementation calendar 3-20. The Task Summary 10-10 further serves as a manipulable navigation link to navigate the Specialist/GP to the Task List page 9-15A.

The Message Summary 10-20 indicates a number of messages that are for the Specialist's/GP's attention. The number indicated by the Message Summary 10-20 may represent unread messages, total messages, flagged messages, and the like. Messages may be received from other members of the Patient's care unit in the form of email-type messages and chat-type messages. Messages may be further received from the control module 1-40, for example, as a result of a trigger condition being detected during analysis of a Patient journal. The Message Summary 10-20 further serves as a manipulable navigation link to navigate the Specialist/GP to the Message/Chat List page 9-16A.

The Patient Summary 10-30 indicates a number of patients currently under the active care of the Specialist/GP. Patients under active care include those who are undergoing treatment, and/or in preparation to undergo treatment, and/or otherwise being managed by the healthcare system 100 and under the responsibility of the Specialist/GP. The Patient Summary 10-30 further serves as a manipulable navigation link to navigate to the Patient Listing 9-11A page.

The Tests Summary 10-40 indicates the number of tests to be reviewed by the Specialist/GP, and/or the number of tests which have been flagged for further actioning by the Specialist/GP, for example by the Specialist/GP or by their nurses. The Tests Summary 10-40 further serves as a manipulable navigation link to navigate to the Test Listing page 9-12.

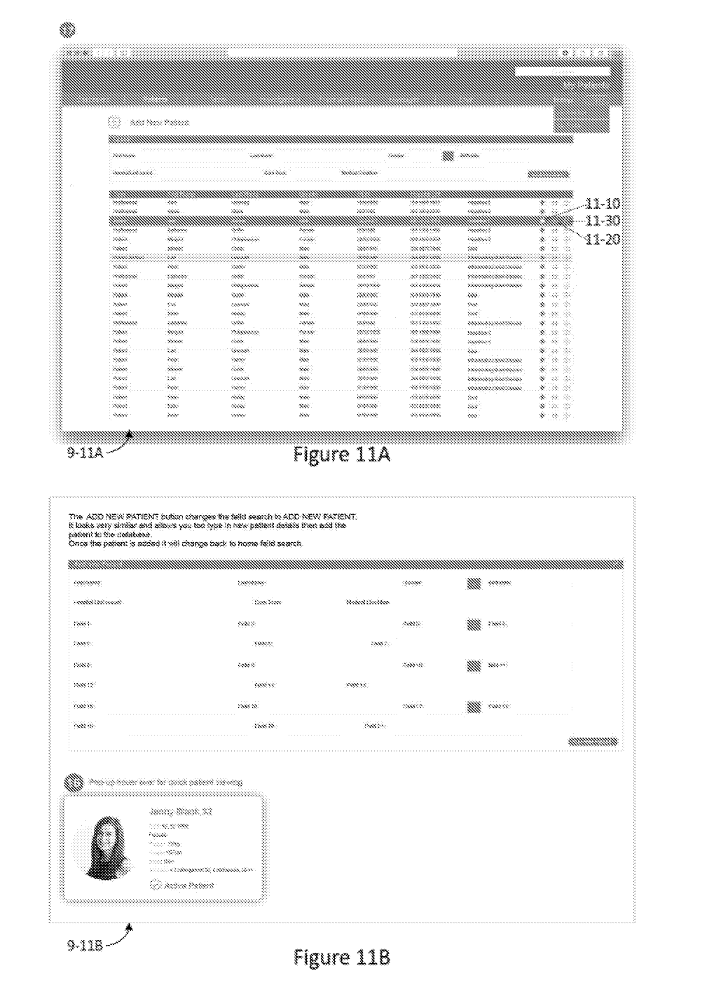

FIGS. 11A to 11C illustrate in greater detail the Patient Listing page 9-11A, Add Patient page 9-11B, and Patient Profile page 9-110.

The Patient Listing page 9-11A lists all patients whose care unit the Specialist/GP is currently (or was) a member of, and further can list all patients who are in some way associated with the Specialist/GP, for example patients who are or have been admitted to a hospital at which the Specialist/GP consults. The Patient Listing page 9-11A includes active patients who are currently being managed through the healthcare system 100, and can also list inactive patients who may not currently be being managed through the healthcare system 100. Details of each patient including first name, last name, gender, date of birth, hospital unit record number, and treated disease are listed. As shown in the right hand columns of 9-11A, functions to chat-message 11-10 and email-message 11-20 each patient are further provided. The selection of such functions takes the Specialist/GP respectively to the Message/Chat list page 9-16A and/or the Chat page 9-16B, as will be described in greater detail below. A further function 11-30 to display more detailed information about any one patient is also provided, the selection of which takes the Specialist/GP to the Patient Profile page 9-110, which is described in greater detail below.

The Add Patient page 9-11B is a portal page that facilitates the addition of a new patient. Details such as the patient's first name, last name, gender, date of birth, hospital unit record number, and present diseases may be entered.

The Patient Profile page 9-110 is a portal page providing detailed information about a patient. Detailed information about a patient includes, for example, all diagnoses for the patient, medication currently or previously taken by the patient, tests performed and corresponding results, current and past treatments, message histories, and the like. The Patient Profile page 9-11C, in the preferred embodiment, is predominantly automatically populated, for example by the control module 1-40 in the course of generating/executing a treatment plan 3-10 and receiving/analysing test results, but may also be manually edited by the Specialist/GP.

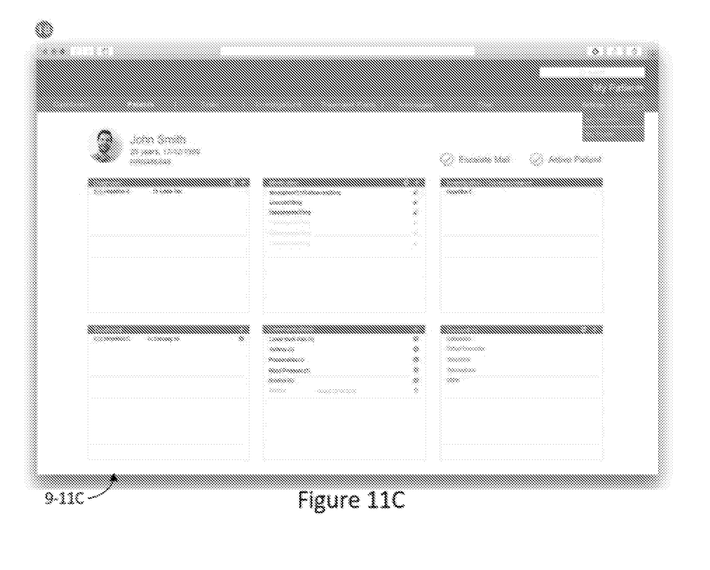

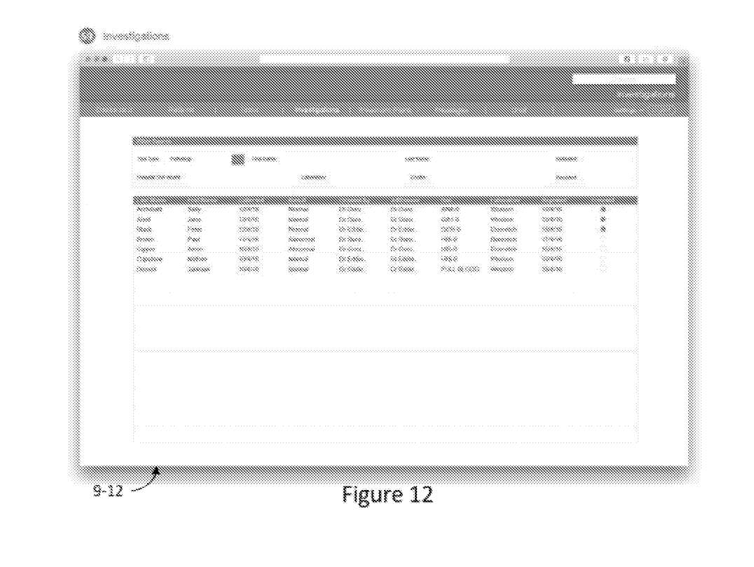

Referring now to FIG. 12, the Test Listing page 9-12 is illustrated and described in greater detail. The Test Listing page 9-12 provides a list of test results matching a given criteria. In a preferred embodiment, the default criterion is that of all tests conducted for all active patients of the Specialist/GP. The test results listed on the Test Listing page 9-12 may additionally or alternatively list all tests:
Of a specified type;
For a specified patient;
Conducted within a specified date range;
With normal results;
With abnormal results;
By a specified laboratory;
For a specified purpose;
By a specified GP/Specialist;
Other criteria; and
A combination of one or more of the above.

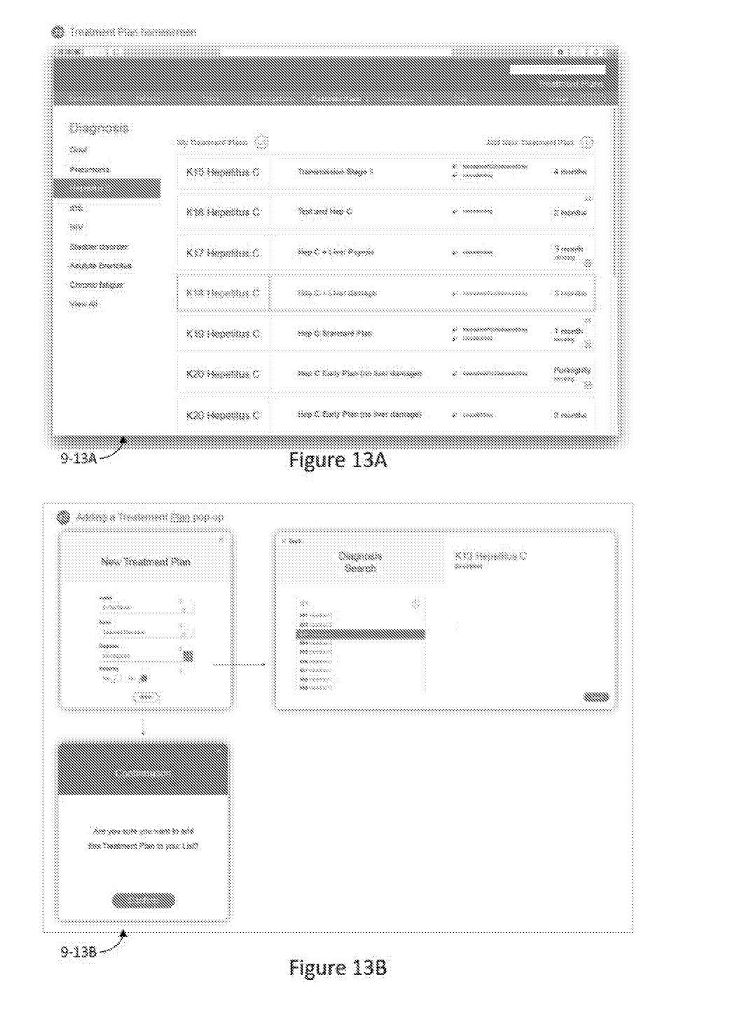

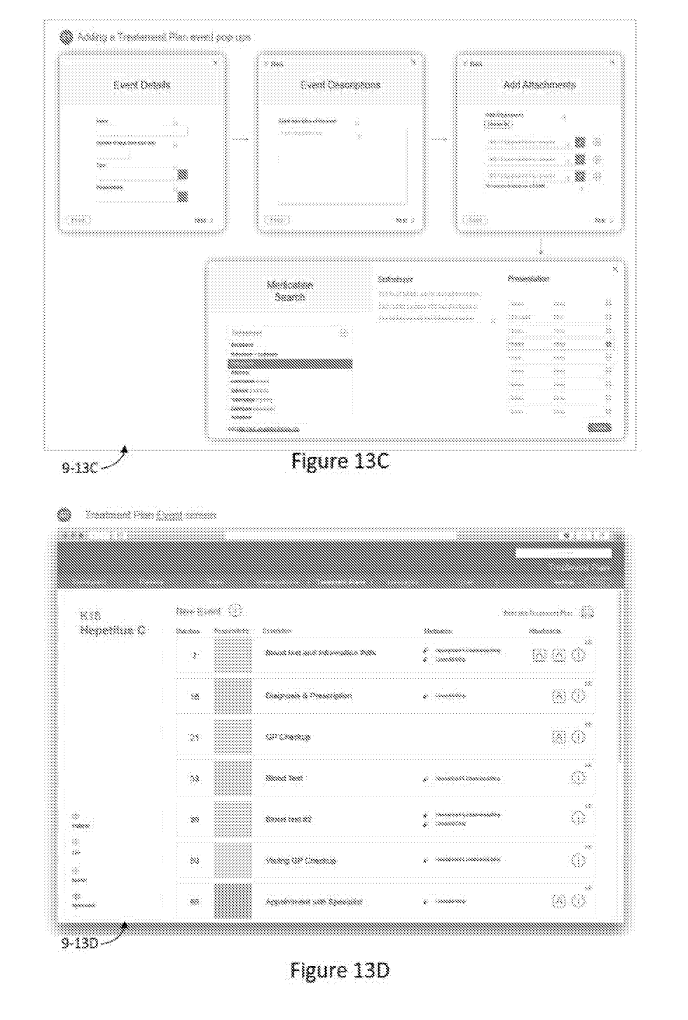

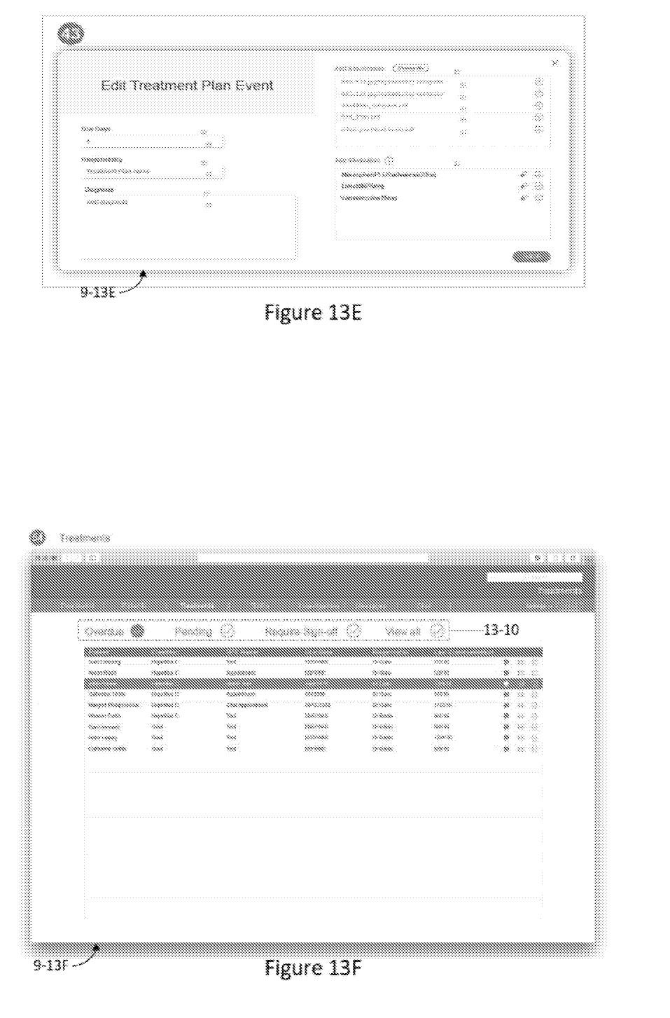

With reference now to FIGS. 13A to 13E the portal pages for managing and creating treatment plan templates 2-10 and treatment plans 3-10 are described.

FIG. 13A illustrates in greater detail the Treatment Plan Template Listing page 9-13A. The Treatment Plan Template Listing page 9-13A lists all treatment plan templates 2-10 available to the Specialist/GP. The treatment plan templates 2-10 may be templates created by the Specialist/GP of the care unit, and/or shared from Specialists/GP from other care units, and/or otherwise imported from other systems. As illustrated, multiple treatment plan templates 2-10 may be listed for each disease, and treatment plan templates for multiple diseases may be available. The Treatment Plan Template Listing page 9-13A, in the illustrated embodiment, lists a name for each treatment plan template 2-10 (e.g. “K18 Hepatitis C”), a description for each treatment plan template 2-10 (e.g. “Hep C+Liver damage”), a drug treatment (e.g. “Linezolid 10 mg”), and a treatment duration (e.g. “3 months”). The information listed on the Treatment Plan Template Listing page 9-13A is not so limited, and may include more or less information than the above.

FIGS. 13B to 13D illustrate in greater detail the Add Treatment Plan Template page 9-13B, Add Event page 9-13C, and Treatment Plan Display page 9-13D, which are used together to create a new treatment plan template 2-10, and in part give effect to general workflow 301 and operation 300.

The Add Treatment Plan Template page 9-13B provides an interface for the Specialist/GP to create a new treatment plan template. Details provided include the Specialists'/GP's name, treatment plan template name, diagnosis to which the treatment plan template is applicable, treatment duration, and the like. Once the details requested by the Add Treatment Plan Template page 9-13B are provided, a blank treatment plan template is created and made available for further editing.

Editing of the blank treatment plan template is facilitated by the Add Event page 9-13C. The Add Event page 9-13C provides an interface for adding events to the blank treatment plan template, thereby populating the blank treatment plan template. Events that may be added to the treatment plan template include, for example:
Initial consultation
Initial drug dosage
Repeat drug dosages
Pathology tests
Patient journal entries
Follow up appointments
Etc.

Responsible and target parties for each event are also specified with each event, so that it is clear who is responsible, accountable, consulted, and/or informed about any particular event, and who the target/subject of the event is.

The treatment plan template 2-10, as it is being populated by events and also once populated, is displayed by the Treatment plan display page 9-13D. Revision of events in the treatment plan template 2-10 is facilitated by the Edit Event page 9-13E.

A list of all treatment plans 3-10 that the Specialist 1-30A has an involvement in, including those for which the Specialist 1-30A is the responsible specialist doctor, as well as those for which the Specialist 1-30A may be consulting for, is listed by the Treatment Plan Listing page 9-13F.

Once population of the blank treatment plan template is complete such that a completed treatment plan template is ready for use, the completed treatment plan template may be published via the Treatment plan display page 9-13D to make the completed treatment plan template available for use.

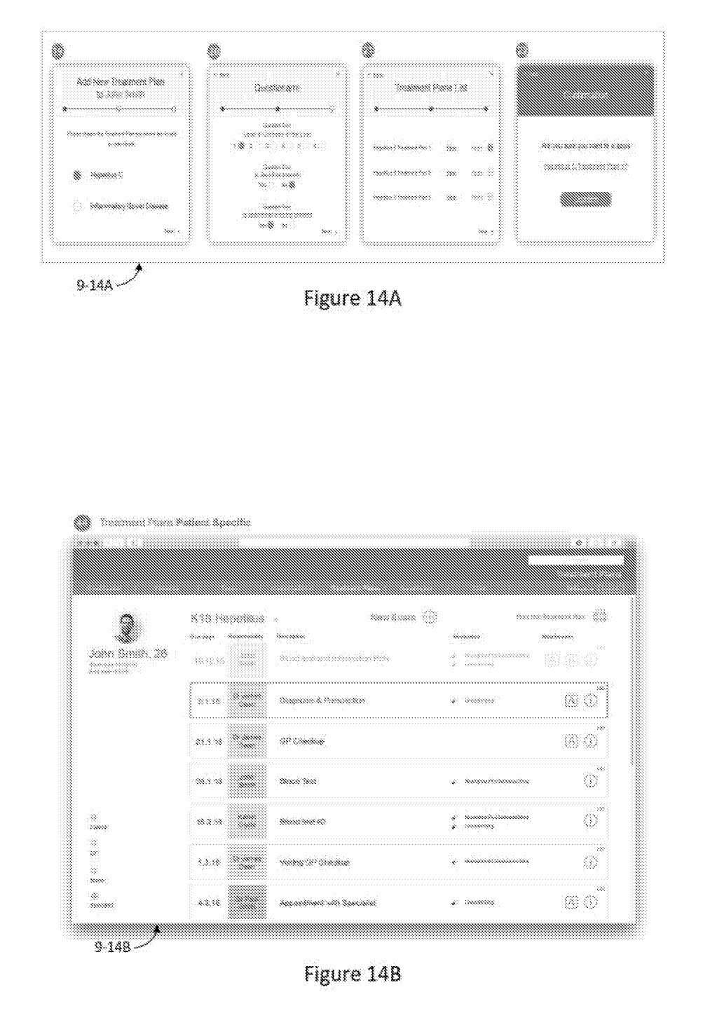

FIGS. 14A and 14B illustrate a Questionnaire page 9-14A and Patient Treatment Plan page 9-14B, which are used to apply a treatment plan template to a Patient 1-30C.

The Questionnaire page 9-14A is an interface presented to the member of a care unit, generally the GP 1-30B or Specialist 1-30A, who is seeking to create a new treatment plan 3-10. The Questionnaire page 9-14A presents a set of predetermined questions to the member, the answers to which, as described previously above, constitute an answer set 2-20 that determines which treatment plan template 2-10 should be applied to the Patient 1-30C.

The Patient Treatment Plan page 9-14B displays the treatment plan 3-10 generated from the treatment plan template 2-10, and specifically applied to the Patient 1-30C. The applied treatment plan 3-10, as illustrated, is displayed by the Patient Treatment Plan page 9-14B with actual dates, responsible parties, and the like, which is in contrast to the treatment plan template 2-10 illustrated in FIG. 13D, where the same information is provided in relative terms rather than as absolute values.

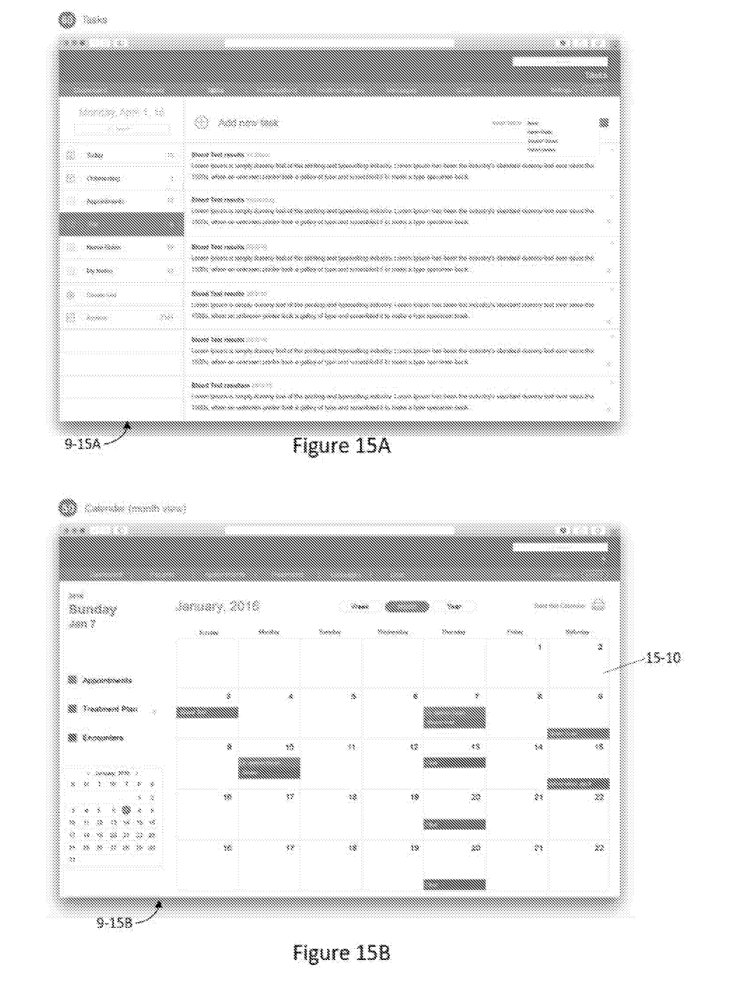

FIGS. 15A and 15B illustrate a task list page 9-15A and calendar display page 9-15B of the Specialist/GP portal. The task list page 9-15A displays a summary of all outstanding tasks for the Specialist/GP. The tasks listed on the task list page 9-15A may include ad hoc tasks created by the Specialist/GP themselves, or by Specialist Nurse/GP Nurse for the Specialist/GP. The tasks listed on the task list page 9-15A can also include tasks generated for the Specialist/GP by the control module 1-40, including for example:
Tasks specified by treatment plan implementation calendars 3-20;
Tasks arising from triggers arising from the analysis of inputs provided to or otherwise obtained by the control module 1-40, for example as part of operation 600.

In the preferred embodiment, a calendar 15-10 displayed by the calendar display page 9-15B may be configured by the user (e.g. Specialist or GP) to display one or more of the following calendar views:
All events;
All events from treatment plans only;
All events from one or more specific treatment plans only;
All events except for events from treatment plans
Appointments from all treatment plans;
Appointments from one or more specific treatment plans only;

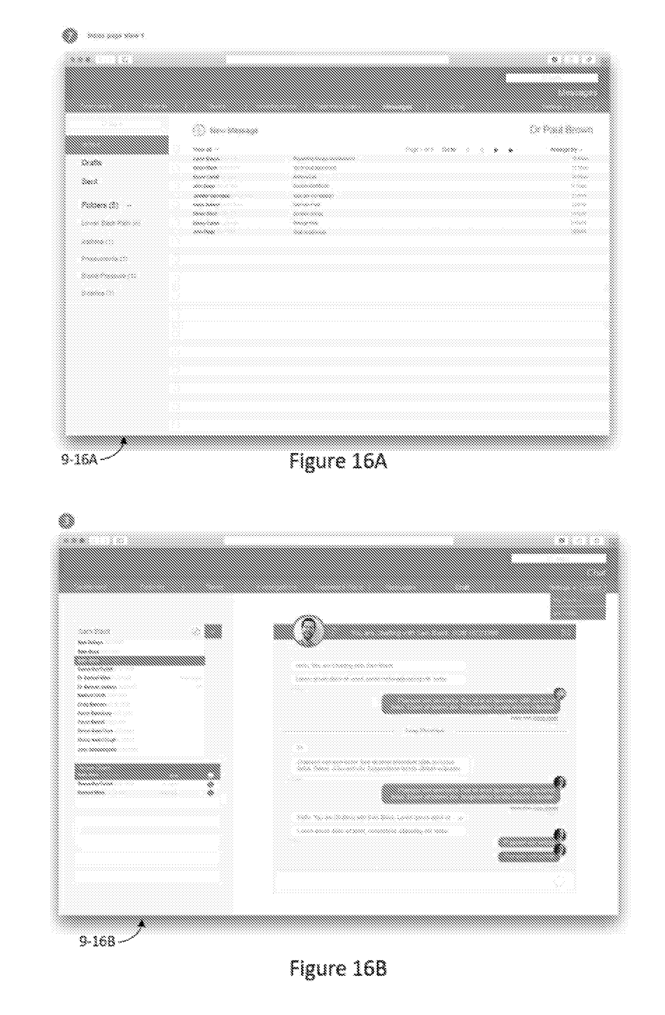

FIGS. 16A and 16B illustrate the Message List page 9-16A and Chat page 9-16B. The message list page 9-16A lists all messages sent to or from the user (e.g. Specialist/GP). The chat page 9-16B shows ongoing chats with members of the care unit. As described above, the Message List page 9-16A and Chat page 9-16B may be accessed also from the chat-message function 12-10 and email-message function 12-20 of the Patient Listing page 9-11A. The Message List page 9-16A and Chat page 9-16B in part give effect to the messaging module 7-40 of the healthcare system 100, and therefore further assists in facilitating and encouraging communication between members of the care unit, while reducing the need for face-to-face appointments.

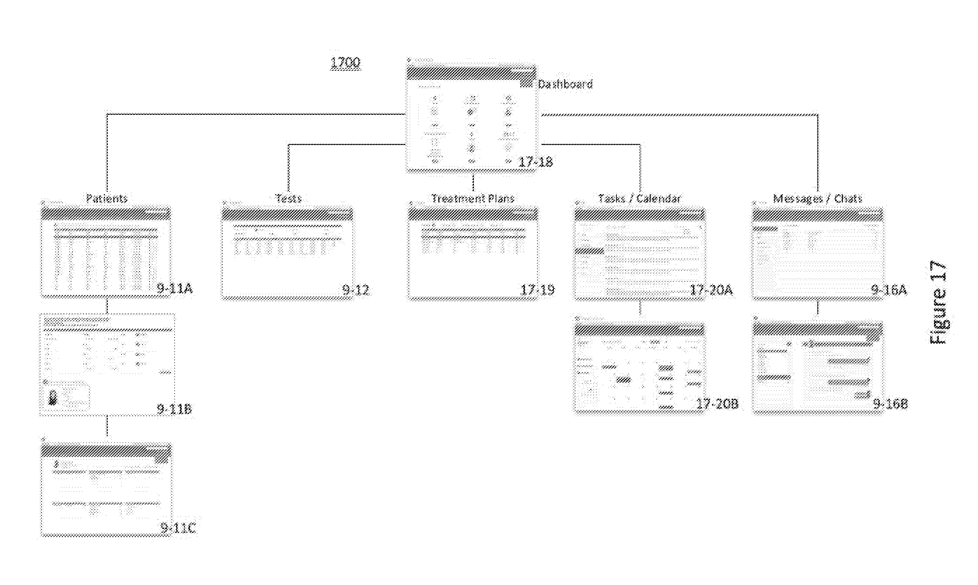

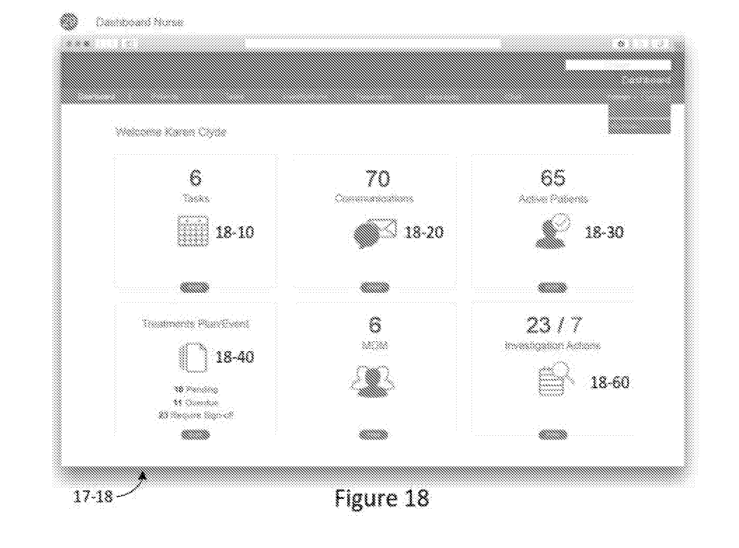

With reference now to FIGS. 17 to 19, the Specialist Nurse portal 1-22B and GP Nurse portal 1-22D (hereinafter referred to as the Nurse portals 1-22BD) will be described in greater detail. The Nurse portals 1-22BD are configured for and made available to the Specialist Nurse 1-30A supporting the Specialist 1-30A, and the GP Nurse 1-30C supporting the GP 1-30C is appropriate.

FIG. 17 illustrates a portal map 1700 of the Nurse portals 1-22BD. The portal map 1700 sets out individual portal pages that make up the Nurse portals 1-22BD. It is to be understood that the portal map 1700 is not limited to only the portal pages illustrated in FIG. 18, and that other portal pages may be added to the portal map 1700 or existing portal pages removed or replaced.

The Nurse portals 1-22BD includes the following portal pages:
Dashboard 17-18;
Patient Listing 9-11A;
Add Patient 9-11B;
Patient Profile 9-110;
Test Listing 9-12;
Treatment Plan Listing 17-19;
Task List 17-20A;
Calendar Display 17-20B;
Message List 9-16A; and
Chat 9-16B

The Patient Listing 9-11A, Add Patient 9-11B, Patient Profile 9-110, Test Listing 9-12, Message List 9-16A and Chat 9-16B pages are essentially identical to those previously describe above with regards to the Specialist/GP portals 1-22A, 1-22C, and provide to the users of the Nurse portals 1-22BD the same functions that those of the Specialist/GP portals 1-22A, 1-22C do to the Specialist/GP.

The Dashboard page 17-18 of the Nurse portals 1-22BD is illustrated in greater detail in FIG. 18. As shown in FIG. 18, the Dashboard page 17-18 provides an overview of the Nurse's outstanding responsibilities, and includes a Task Summary 18-10, Message Summary 18-20, Patient Summary 18-30, Treatment Summary 18-40, and Test Summary 18-60.

The Task Summary 18-10, Message Summary 18-20, Patient Summary 18-30, and Test Summary 18-40 perform similar functions to those of the Task Summary 10-10, Messages Summary 10-20, Patient Summary 10-30, and a Test Summary 10-40 of Dashboard 9-10.

The Treatment Summary 18-40 provides an indication treatment plan items requiring the Nurse's attentions, including for example, draft treatment plans that are pending a Specialist's/GP's authorization, overdue treatment plan events (which may also be displayed in the Task Summary 18-10), proposed revisions to treatment plans 3-10 and/or treatment plan implementation calendars 3-20, and the like. The Treatment Summary further serves as a manipulable navigation link to navigate the Nurse to the Treatment Plan Listing page 17-19.

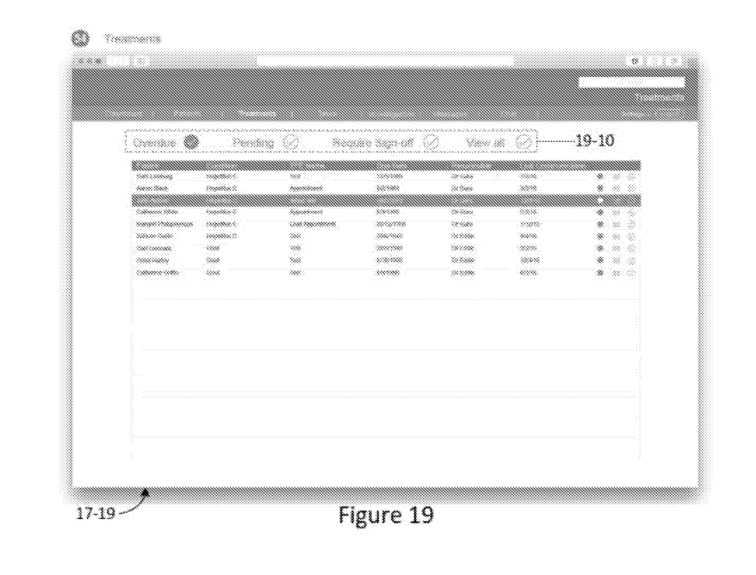

FIG. 19 illustrates in greater detail the Treatment Plan Listing page 17-19. The Treatment plan listing page 17-19 of the Nurse portals 1-22BD differs from that of the Specialist/GP portal 1-22A, 1-22C in that the Treatment plan listing page 17-19 provides only a listing of draft and applied treatment plans 3-10, and the Patients 1-30C to which they are applied or to be applied. In contrast to the Specialist/GP portal 1-22C, creation of a new draft treatment plan is generally not permitted, though such function may be added if advantageous to a particular care unit. Additionally, the Treatment Plan Listing page 17-19 of the Nurse portals 1-22BD includes filter functions 19-10 for filtering the list of treatment plans so as to show only treatment plans that are, for example, overdue, pending, require sign-off, are the subject of proposed revisions, and the like. Further, the Treatment Plan Listing page 17-10 of the Nurse portals 1-22BD list all treatment plans 3-10 of Specialists or GPs whom the nurses (that is, the GP Nurse or Specialist Nurse) are supporting.

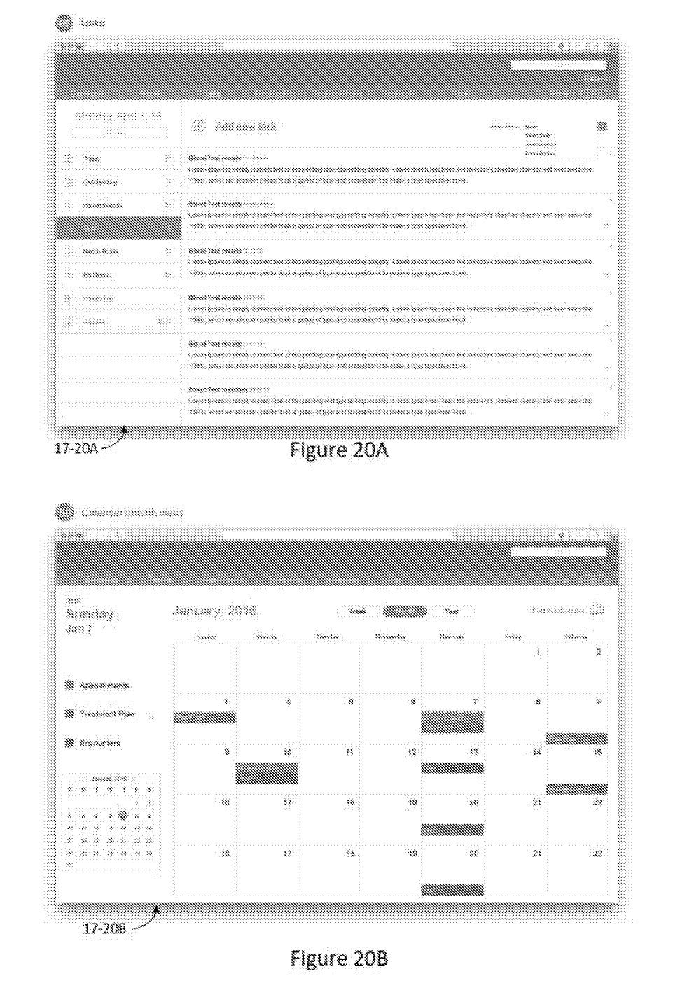

FIGS. 20A and 20B illustrate the Task List page 17-20A and Calendar display page 17-20B of the Nurse portals 1-22BD. The Task List page 17-20A and Calendar display page 17-20B of the Nurse portal 1-22 differ from those of the Specialist/GP portal 1-22C in that these pages in the Nurse portal 1-22 allow the user of the Nurse portal 1-22 to view not just tasks and events assigned to the Specialist Nurse or GP Nurse, but also to one or more other members of the care unit. In the preferred embodiment, the Task list page 17-20A and Calendar display page 17-20B of the Nurse portal 1-22 provides visibility to the tasks, events, appointments, reminders, notifications, and any other piece of information, that would appear on a task list and calendar of any member of the care unit.

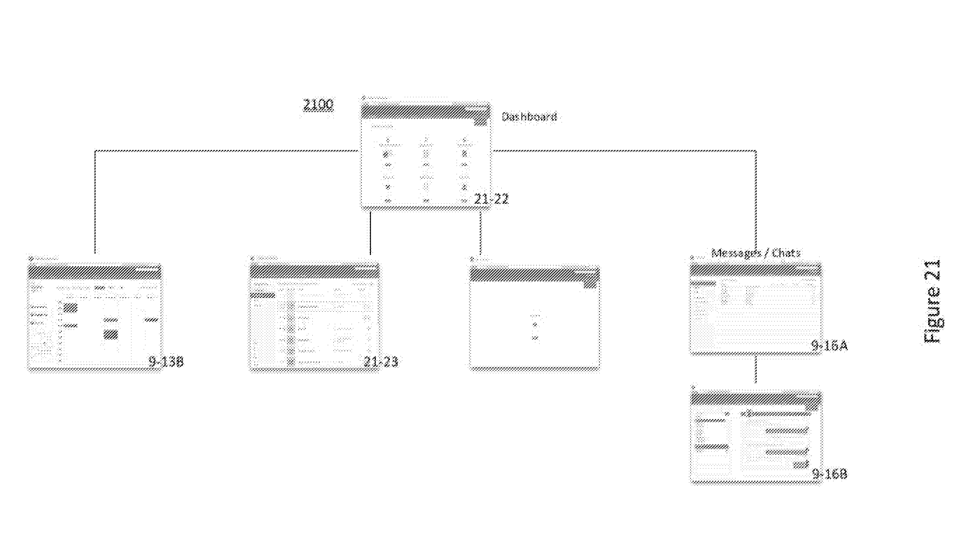

With reference now to FIGS. 21 to 23, the Patient portal 1-22E and Carer portal 1-22F will be described in greater detail. The Patient portal 1-22E and Carer portal 1-22F are configured for and made available respectively to the Patient 1-30C and Patient's Carer 1-30C.

FIG. 21 illustrates a portal map 2100 of the Patient/Carer portals 1-22E, 1-22F. The portal map 2100 sets out individual portal pages that make up the Patient/Carer portals 1-22E, 1-22F. It is to be understood that the portal map 2100 is not limited to only the portal pages illustrated in FIG. 21, and that other portal pages may be added to the portal map 2100 or existing portal pages removed or replaced.

The Patient/Carer portals 1-22E, 1-22F include the following portal pages:
Dashboard 21-22;
Calendar Display 9-13B;
Treatment Plan 21-23;
Message List 9-16A; and
Chat 9-16B.
Patient journal
Health record
Care unit

The Calendar Display page 9-13B9-11A, Message List page 9-16A, and Chat page 9-16B are essentially identical to those previously describe above with regards to the Specialist/GP portals 1-22A, 1-22C, and provide to the users of the Patient/Carer portals 1-22E, 1-22F the same functions that those of the Specialist/GP portals 1-22A, 1-22C do to the Specialist/GP.

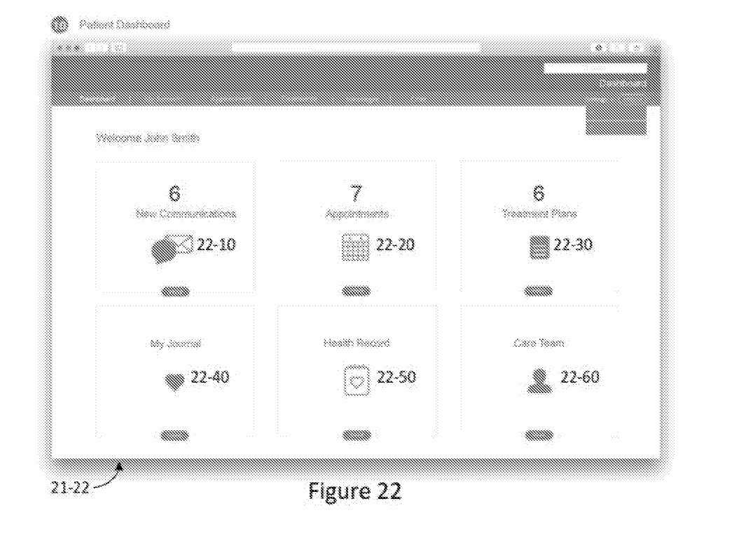

The Dashboard page 21-22 of the Patient/Carer portals 1-22E, 1-22F is illustrated in greater detail in FIG. 22. As shown in FIG. 22, the Dashboard page 21-22 provides an overview of the Patient's outstanding responsibilities, and includes a Communication Summary 22-10, Appointment Summary 22-20, Treatment Plan Summary 22-30, Patient Journal 22-40, Health Record 22-50, and Care Unit Details 22-60.

The Communication Summary 22-10 provides an indication of the number of email-messages or chat-messages marked for the attention of the Patient 1-30C, and when interacted with, navigates the Patient 1-30C to the Message List page 9-16A. The number indicated by the Communication summary 22-10 may refer to a number of unread/new messages, number of total messages, number of flagged messages, and the like.

The Appointment Summary 22-20 provides an indication of the number of appointments scheduled for the Patient 1-30C. The indicated number may be of appointments for the current day, week, month, or for all future appointments. Interacting with the Appointment Summary 22-20 navigates the Patient 1-30C to the Calendar Display page 9-13B.

The Treatment Plan Summary 22-30 provides an indication of all treatment plans that the Patient 1-30C is currently on. Interaction with the treatment plan summary 22-30 navigates the Patient 1-30C to the treatment plan page 21-23.

The Patient journal 22-40 navigates the Patient 1-30C to a journal page, where the Patient 1-30C can view past journal entries, view and update specific journals assigned to the Patient 1-30C by members of the care unit, and/or view and update the general patient journal.

The Health Record 22-50 navigates to a Patient Health Record page which lists all confirmed diagnoses of the patient, prescribed medication, medication dosages/frequency, and other information summarising a medical history of the Patient 1-30C. The Health Record 22-50 serves as a record of information that belongs to and follows the Patient 1-30C, thereby allowing future and different care units caring for the Patient 1-30C to have access to the Patient's medical history simply by virtue of the Patient 1-30C being a user of the healthcare system 100.

The Care Unit 22-60 navigates to a Care Unit Profile page where details of each member of each of the Patient's care units are listed.

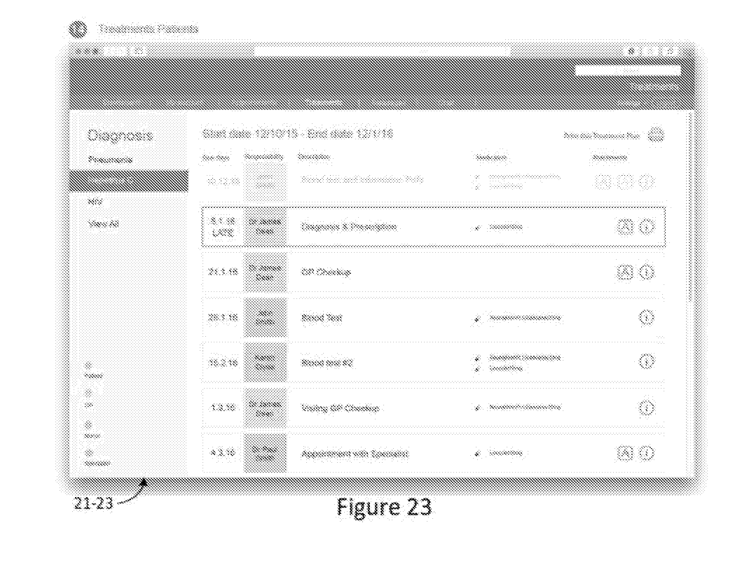

The Treatment Plan page 21-23 of the Patient/Carer portals 1-22E, 1-22F is illustrated in greater detail in FIG. 23. As illustrated, the Treatment Plan page 21-23 lists all treatment plans that the Patient 1-30C is on, and further lists the details of the treatment plan such as events, medication, and other information such as those provided by attachments.

As described above, the present invention provides a healthcare system 100 that allows for the care, treatment, and management of a patient with a chronic disease to be undertaken remotely, with a greater degree of automation, with less reliance on the Specialist 1-30A, greater oversight by the Specialist Nurse 1-30A, and therefore with greater compliance by the Patient 1-30C.

By way of the healthcare system 100, and in particular the various portals 1-20, a draft treatment for a specialist-treated disease may be created with a relatively high level of competency by a member of the care unit other than the Specialist 1-30A, through the use of treatment plan templates 2-10, question sets 2-15, and answer sets 2-20 created by the Specialist 1-30A; thereby reducing the time burden on the Specialist 1-30A. Furthermore, in facilitating the review and subsequent authorization of draft treatment plans by the Specialist 1-30A, the quality of care provided to the Patient 1-30C is not diminished by the fact that the draft treatment plan is created by a member of the care unit other than the Specialist 1-30A.

The automated generation of user specific treatment plan implementation calendars 3-20 ensures greater adherence and compliance to the treatment plan 3-10 by the Patient 1-30C, and further ensures better, more error-free execution of the treatment plan 3-10 by the Specialist 1-30A, Specialist Nurse 1-30A, GP 1-30B, GP Nurse 1-30B, and/or other Profession 1-30D; whilst at the same time reducing the administrative burden on the care unit, in particular the Specialist Nurse 1-30A and GP Nurse 1-30B.

The ability to specify search parameters for tests, and for the healthcare system 100 (specifically the control module 1-40) to then automatically and periodically search for matching tests allows superfluous, redundant, and unnecessary tests to be performed on the Patient 1-30C, thereby saving time, and costs.

The ability to specify analysis parameters to analyse test results, data from health sensors, information provided by the Patient 1-30C through the patient journal and other means, and for the healthcare system 100 (specifically the control module 1-40) to then automatically analyse such information according to the analysis parameters and execute pre-designated responses allows a higher level of healthcare to be provided, and for more effective disease treatment. This ultimately results also in time and cost savings.

### Advantages

The preferred embodiment of the present invention allows the time of healthcare professionals involved in the care, treatment, and management of a patient with a chronic disease, in particular the Specialist Doctor, to be more effectively utilized. The preferred embodiment of the present invention, in particular, allows for tasks, which conventionally have been performed largely by the Specialist Doctor, to be shifted from the Specialist Doctor to other members of a patient's care unit, including the General Practitioner Doctor, Specialist Nurse, and the patient him/herself. This allows more effective triaging of stable patients to be managed by the GP and Specialist nurse, and allows the Specialist Doctor to focus on the treatment and care of more complex chronic patients. The preferred embodiment of the present invention further allows the Specialist to remain engaged with not only the patients in the care of the care unit, but also members of the care unit themselves. The preferred embodiment of the present invention allows the same, if not higher, level of care to be provided to stable chronic patients, and the same, if not higher, level of compliance with a treatment plan to be achieved by the Patient compared to the current model of health care (e.g. the Australian health care system as at 2016). Moreover, efficiencies in the care, treatment, and management of a patient with a chronic disease are gained through a reduction in duplication of work and reduction in unnecessary and/or mistimed appointments. Overall, the preferred embodiment of the present invention significantly reduces strain on the health system, and ultimately reduces costs.

### Variations

While the foregoing has been given by way of illustrative example of this invention, all such and other modifications and variations thereto as would be apparent to persons skilled in the art are deemed to fall within the broad scope and ambit of this invention as is herein set forth.

Throughout the description and claims of this specification the word “comprise” and variations of that word such as “comprises” and “comprising”, are not intended to exclude other additives, components, integers or steps.

1. A healthcare provision and management system, comprising:
a first electronic device, the first electronic device configured and operable to receive information about a patient;
a server, the server storing therein one or more treatment plan templates and configured and operable to receive the information from the first electronic device over a network;
a control module, the control module being included in, and/or forming a part of, the server, the control module being configured to control the server to analyse the received information and, based on the received information, identify a treatment plan template from which to generate a draft treatment plan for the patient; and
a second electronic device, the second electronic device configured and operable to receive the draft treatment plan and review, revise, and/or authorise the draft treatment plan to produce a finalized treatment plan, wherein
the control module is further configured to control the server to analyse the finalized treatment plan and generate therefrom one or more treatment plan implementation calendars each containing one or more events.

2. A system as claimed in claim 1, further comprising a third electronic device, the third electronic device configured and operable to periodically receive from the patient further information about the patient and transmit this further information to the server.

3. A system as claimed in claim 2, further comprising a fourth electronic device, the fourth electronic device configured and operable to review and revise the one or more treatment plan implementation calendars.

4. A healthcare provision and management system, comprising:
a first electronic device, the first electronic device configured and operable to receive information about a patient;
a server, the server configured and operable to receive the information from the first electronic device over a network;
a control module, the control module being included in, and/or forming a part of, the server, the control module being configured to control the server to analyse the received information and, based on the received information, generate a draft treatment plan for the patient; and
a second electronic device, the second electronic device configured and operable to receive the draft treatment plan and review, revise, and/or authorise the draft treatment plan to produce a finalized treatment plan, wherein
the control module is further configured to control the server to analyse the finalized treatment plan and generate therefrom one or more treatment plan implementation calendars each containing one or more events, wherein:
the information received by the first electronic device includes answers provided in response to a specific question set, and the first electronic device is configured to generate an answer set from the answers;
the answer set represents a combination of answers which identifies and is logically associated with a treatment plan template; and
the control module generates the draft treatment plan using the associated treatment plan template.

5. A system as claimed in claim 2, wherein the further information is received by the third electronic device from health sensors monitoring the patient, or by way of journal entries entered by the patient through the third electronic device.

6. A system as claimed in claim 3, wherein the first electronic device is operated by a general practitioner doctor, the second electronic device is operated by a specialist doctor, the third electronic device is operated by the patient, and the fourth electronic device is operated by a specialist nurse, and the control module is configured to generate a treatment plan implementation calendar that is specific for each of the first, second, third, and fourth electronic devices.

7. A system as claimed in claim 6, wherein one or more of the first, second, and fourth electronic devices is operable to generate an analysis parameter for analysing the further information about the patient, and the control module is operable to monitor the further information about the patient and determine if any of the further information satisfies the analysis parameter.

8. A system as claimed in claim 7, wherein the control module performs a triggered revision of one or more of the treatment plan implementation calendars upon determining that a piece of the further information satisfies the analysis parameter.

9. A system as claimed in claim 7, wherein the control module sends a message or notification to one or more of the first, second, and fourth electronic devices upon determining that a piece of the further information satisfies the analysis parameter.

10. A system as claimed in claim 6, wherein one or more of the first, second, and fourth electronic devices is operable to generate a search parameter for searching for test results or test requests, and the control module is operable to search one or more databases for test results or test requests satisfying the search parameter, and wherein the control module revises one or more treatment plan implementation calendars upon identifying a test result or test request satisfying the search parameter.

11. A system as claimed in claim 6, wherein the treatment plan implementation calendar specific for the first electronic device is generated to include one or more of:
(a) Dates and timings for checking in with the patient;
(b) Dates and timing for receiving reports from one or more of the specialist doctor, patient, or specialist nurse;
(c) Dates and timings for sending reports to one or more of the specialist doctor, patient, or specialist nurse;
(d) Dates and timings of patient appointments;
(e) Dates and timings of test results due from the patient;
(f) Dates and timings for the issuance of medication prescription scripts;
(g) Dates and timings for the issuance of test requests;
(h) Partially pre-filled draft prescription scripts; and
(i) Partially pre-filed test requests.

12. A system as claimed in claim 6, wherein the treatment plan implementation calendar specific for the second electronic device is generated to include one or more of:
(a) Dates and timings for checking in with the patient;
(b) Dates and timing for receiving reports from one or more of the specialist doctor, patient, or specialist nurse;
(c) Dates and timings for sending reports to one or more of the specialist doctor, patient, or specialist nurse;
(d) Dates and timings of patient appointments;
(e) Dates and timings of test results due from the patient;
(f) Dates and timings for the issuance of medication prescription scripts;
(g) Dates and timings for the issuance of test requests;
(h) Partially pre-filled draft prescription scripts; and
(i) Partially pre-filed test requests.

13. A system as claimed in claim 6, wherein the treatment plan implementation calendar specific for the third electronic device is generated to include one or more of:
(a) Dates and timings for drug doses;
(b) Information regarding drugs;
(c) Dates and timings for tests;
(d) Dates and timings for filling medication scripts;
(e) Dates and timings of appointments with the specialist doctor, specialist nurse, and/or general practitioner doctor;
(f) Dates, timings and contents of entries to be made into one or more patient journals;
(g) Prescription scripts; and
(h) Test requests.

14. A system as claimed in claim 6, wherein the treatment plan implementation calendar specific for the fourth electronic device is generated to include one or more of:
(a) Dates and timings for checking in with the patient;
(b) Dates and timing for receiving reports from one or more of the specialist doctor, patient, or general practitioner doctor;
(c) Dates and timings for sending reports to one or more of the specialist doctor, patient, or general practitioner doctor;
(d) Dates and timings of patient appointments;
(e) Dates and timings of test results due from the patient;
(f) Dates and timings for the issuance of medication prescription scripts;
(g) Dates and timings for the issuance of test requests;
(h) Partially pre-filled draft prescription scripts; and
(i) Partially pre-filed test requests.

15. A system as claimed in claim 1, wherein:
the first electronic device is further operable to generate a treatment plan template, a question set, and one or more answer sets which each represent one combination of answers to the question set, and
the server is operable to store the treatment plan template in association with at least one of the answer sets, such that the associated answer set(s) identify the treatment plan template.

16. A system as claimed in claim 6, wherein the finalized treatment plan includes an event for setting up a patient journal, and the control module is operable to set up a patient journal accessible by the third electronic device and adapted for input into thereby.

17. A system as claimed in claim 16, wherein the event for setting up the patient journal is pre-specified by a treatment plan template on which the finalized treatment plan is based.

18. A system as claimed in claim 16, wherein the event for setting up the patient journal is added to the finalized treatment plan by the control module in response to an analysis conducted by the control module on the further information about the patient.

19. A healthcare provision and management method, the method comprising:
receiving information about a patient;
analysing the received information and, from the analysis of the received information, automatically identifying a treatment plan template from which to generate a draft treatment plan for the patient;
generating a draft treatment plan from the identified treatment plan template;
reviewing, revising, and/or authorising the draft treatment plan to produce a finalized treatment plan; and
analysing the finalized treatment plan to automatically generate therefrom one or more treatment plan implementation calendars each containing one or more events.

20. A method as claimed in claim 19, further comprising periodically receiving further information about the patient, and reviewing and/or revising the one or more treatment plan implementation calendars in response to the received further information.

21. A method as claimed in claim 20, wherein the further information is received from health sensors monitoring the patient, or by way of journal entries entered by the patient.

22. A method as claimed in claim 19, wherein the one or more automatically generated treatment plan implementation calendars are each generated to be specific for a GP, Specialist, Specialist Nurse, and Patient making up a care unit of the Patient.

23. A method as claimed in claim 21, further comprising generating an analysis parameter for analysing the further information about the patient and monitoring the further information about the patient to determine if any of the further information satisfies the analysis parameter.

24. A method as claimed in claim 23, further comprising performing a revision of one or more of the treatment plan implementation calendars upon determining that a piece of the further information satisfies the analysis parameter.

25. A method as claimed in claim 23, further comprising sending a message or notification upon determining that a piece of the further information satisfies the analysis parameter.

26. A method as claimed in claim 21, further comprising generating a search parameter for searching for test results or test requests, and searching one or more databases for test results or test requests satisfying the search parameter.

27. A method as claimed in claim 26, further comprising revising one or more treatment plan implementation calendars upon identifying a test result or test request satisfying the search parameter.

28. A method as claimed in claim 19, wherein the finalized treatment plan includes an event for setting up a patient journal, and method further comprises automatically setting up the patient journal at the date specified by said event, and wherein the event for setting up the patient journal is pre-specified by the treatment plan template on which the finalized treatment plan is based, or the event for setting up the patient journal is added to the finalized treatment plan by the control module in response to an analysis conducted on the further information about the patient.

29. A method as claimed in claim 28, further comprising automatically analysing inputs into the patient journal according to one or more analysis parameters.
